(19) United States
(12) Patent Application Publication
(10) Pub. No.: US 2013/0137593 A1
(43) Pub. Date: May 30, 2013

(54) EARLY DETECTION AND STAGING OF COLORECTAL CANCER USING A PANEL OF MICRO RNAS

(71) Applicants: Aviram Nissan, Ganei Yehuda (IL); Stella Mitrani Rosenbaum, Jerusalem (IL); Nadia Ilyayev, Bet Shemesh (IL); David Halle, Efrat (IL)

(72) Inventors: Aviram Nissan, Ganei Yehuda (IL); Stella Mitrani Rosenbaum, Jerusalem (IL); Nadia Ilyayev, Bet Shemesh (IL); David Halle, Efrat (IL)

(73) Assignee: HADASIT MEDICAL RESEARCH SERVICES AND DEVELOPMENT LTD., Jerusalem (IL)

(21) Appl. No.: 13/641,308

(22) PCT Filed: Apr. 14, 2011
(86) PCT No.: PCT/IL11/00310

§ 371 Date: Feb. 5, 2013

Related U.S. Application Data
(60) Provisional application No. 61/324,494, filed on Apr. 15, 2010.

(51) Int. Cl. C12Q 1/68

(52) U.S. Cl. CPC C12Q 1/6886
USPC 506/9; 435/6.11; 435/6.12

(57) ABSTRACT

The present invention provides compositions, methods and kits for diagnosing cancer, specifically the diagnosis of colorectal cancer (CRC). More specifically, the invention provides simple assays, with high sensitivity and specificity for CRC, wherein a panel of microRNA (miRNA) are used as biomarkers.

### FIELD OF THE INVENTION

The present invention is directed to the field of cancer diagnosis and staging, specifically of colorectal cancer. In particular, the invention provides compositions, methods and diagnostic kits using a panel of microRNA molecules.

### BACKGROUND OF THE INVENTION

Colorectal cancer (CRC) is the fourth most common cancer and second leading cause of cancer-related death in the US, with more than 140,000 new cases diagnosed annually (Jemal A et al. CA Cancer J Clin 2009: 59; 225-249). In Western countries, adenocarcinoma of the colon and rectum accounts for more new cases of cancer per year than any anatomic site except the lung.

Features of malignant adenocarcinoma, distinct from benign tumors, include invasion and metastasis. Malignant tumors are fatal, mostly due to their capacity to invade neighboring tissues and metastasize through the lymphatic system and bloodstream to nearby or distant organs.

The survival and prognosis of CRC patients depends mainly on the disease stage at the time of detection. Global 5-year survival of patients without lymph node involvement is about 80% and drops if positive lymph nodes or distant metastasis are detected (stage 3 and 4) (Bosch Roig et al., Clin Transl Oncol 2008: 10, 572-278; Schepeler et al., Cancer Res 2008: 68(15); 6416-6424). Precise determination of the regional lymph nodes status is the most important diagnostic and prognostic factor in surgically resectable colorectal adenocarcinoma and defines the need for adjuvant chemotherapy (Bosch Roig et al. ibid; Aslam et al., British Jour. of Surgery 2009: 96; 702-710).

Currently, most CRC patients are diagnosed by their symptoms which become apparent usually when the disease is at a relatively advanced stage and requires surgery and adjuvant therapy. The ability to use specific biomarkers to screen for CRC, for example in blood or stool samples, can improve early detection and may prevent invasive cancer if the patient is diagnosed at an early stage.

Early detection and prevention of CRC are feasible. The process of CRC tumorigenesis starting at the mucosa (the innermost layer of the colon) and slowly progressing through a pre-malignant phase (adenomatous polyp) into an invasive cancer provides an excellent opportunity for early detection and prevention of this disease.

The most common non-invasive test for diagnosing CRC is the fecal occult blood test (FOBT). Unfortunately, in addition to its high false-positive rate, the sensitivity of the FOBT remains around 30%-50% and may not detect early malignancy, since not all carcinomas shed blood in the early phase of their development.

Fiber-optic colonoscopy can potentially detect the vast majority of adenomas and carcinomas of the colon. Therefore, it is considered to be the gold standard for detecting colon cancer. However, despite its accuracy, fiber-optic colonoscopy is currently used to screen high-risk populations (e.g. individuals with genetic predisposition and/or family history) and its application to large population screening has failed mainly due to low compliance, high-costs and complications associated with the procedure. FOBT followed by fibro-optic colonoscopy (in FOBT positive cases) was shown to significantly reduce CRC-related mortality.

Exfoliation of normal colonic cells and cells that have undergone malignant transformation is the basis for molecular-based stool assays for early detection of CRC. These assays are based on somatic mutations characteristic of CRC and on epigenetic changes found in CRC. The variety of molecular events resulting in a phenotype of colonic adenoma or carcinoma limits these assays to detection of tumors harboring the mutations or hypermethylated genes examined while other tumors may go undetected.

Numerous serum markers, such as carcinoembryonic antigen (CEA), carbohydrate antigen 19-9, and lipid-associated sialic acid, have been investigated in colorectal cancer, but their low sensitivity has limited their role, as reflected by the American Society of Clinical Oncology (ASCO) guidelines, to monitoring therapy and for post-therapy surveillance. Currently, none of the molecular markers, including CEA and CA-19-9, are recommended for screening and diagnosis.

Surgical resection is very effective treatment for patients with localized tumors, however approximately 20-25% of patients who are diagnosed as lymph node negative, by conventional histopathological methods will develop recurrence and will die of the disease (Bilchick et al., Annals of Surgery 2007: 246; 568-577). The high rate of recurrence may be attributed to the presence of occult lymph node metastases undetected by conventional histopathology or due to minimal residual disease (MRD) in the form of circulating tumor cells in the blood, lymphatics or peritoneal cavitiy (Nissan, Journal of Surgical Oncology 2007: 96; 185-187). Addition of serial sectioning and immunohistochemical cytokeratine analysis (IHC) to standard sectioning and E&H staining, improve staging accuracy in 4-39% of patients. RT-PCR technique for tumor molecular markers increases lymphatic staging sensitivity by 15-50% (Bosch Roig et al. ibid; Stojadinovic and Nissan et al., Annals of Surgery 2007; 245(6): 846-857).

### MicroRNA

MicroRNAs (miRNAs) are a large class of single strand RNA molecules of 18-25 nucleotides, involved in post transcriptional gene silencing. Eighty percent of conserved miRNA show tissue-specific expression and play an important role in cell fate determination, proliferation, and cell death (Lee and Dutta. Annu. Rev. Pathol. Mech. Dis. 2009; 4: 199-227; Ross, Carlson and Brock, Am J Clin Path 2007: 128; 830-836). miRNAs arise from intergenic or intragenic (both exonic and intronic) genomic regions that are transcribed as long primary transcripts (pri-microRNA) and undergo a number of processing steps to produce the final short mature molecule (Massimo et al., Current Op. in Cell Biol. 2009: 21; 1-10).

The mature miRNAs suppress gene expression based on their complementarity to a part of one or more mRNAs usually in the 3′ UTR site. The annealing of miRNA to the target transcript either blocks protein translation or destabilizes the transcript and triggers the degradation or both. Most of the miRNA action on target mRNA translation is based on the partial complementarity, therefore conceivably one miRNA may target more than one mRNA and many miRNAs may act on one mRNA (Ying at el., Mol. Biotechnol. 2008: 38; 257-268). In humans, approximately one-third of miRNAs are organized into clusters. A given cluster is likely to be a single transcriptional unit, suggesting a coordinated regulation of miRNAs in the cluster (Lee and Dutta. ibid).

Although the biological functions and the target genes of miRNAs are yet to be characterized, there is growing evidence to indicate involvement of miRNAs in the pathogenesis of human cancers. The main mechanism of miRNome alterations in cancer cells is aberrant gene expression, which is characterized by abnormal expression levels of mature miRNAs (Massimo et al. ibid). Large scale studies suggest that a deregulated miRNA profile mostly arises from epigenetic regulation of miRNA expression, abnormal miRNA processing or frequently location (about 50%) of miRNA genes in CAGRs (cancer-associated genomic regions) (Rossi et al., Mamm Genome 2008: 19(7-8); 526-540). A growing amount of evidences provides that miRNAs can act as oncogenes (oncomiRs) that activating the malignant potential by targeting tumor suppressors or tumor suppression genes (Garzon et al., Trends in Mol. Med. 2006: 12(12); 580-587). MicroRNA expression analysis indicated that in addition to differences of miRNA expression between tumor and normal tissues, there are unique characterizing miRNA signatures associated with diagnosis, prognosis, cancer staging, cancer progression and other clinical variables. Consequently, miRNAs can be used as diagnostic or prognostic molecular markers to classify cancer (Galin and Croce, Nat Rev Cancer 2006: 6; 857-866).

Unlike messenger RNA, microRNAs are less likely to be degraded by RNases and can be retrieved intact in the serum or in stool samples. This is of great advantage for using microRNAs, specifically a CRC-related microRNA panel for detection of CRC.

Methods for detecting and/or monitoring colorectal cancer by observing changes in the expression of selected miRNAs are disclosed, inter alia, in WO 10/058,393, WO 10/004,562, WO 09/111,643, WO 09/140,670, WO 09/059,026 and WO 05/118806, and in US Patent Application Publication No. US 2009/0263803.

No where in the art is it disclosed that a specific set of miRNAs may be used for reliable early diagnosis and staging of colorectal cancer, particularly as biomarkers having high specificity and sensitivity for diagnosing occult metastasis. In particular, no set of miRNAs has been developed for successfully diagnosing pre-cancerous polyps.

There remains an unmet need for improved compositions and methods for providing early diagnosis of colorectal cancer and for determining disease staging and prognosis.

### SUMMARY OF THE INVENTION

The present invention provides compositions, methods and kits for diagnosing colorectal cancer (CRC), particularly the diagnosis of micrometastatic disease and/or occult metastasis of CRC tumors. More specifically, the invention provides simple assays, with high sensitivity and specificity for CRC, wherein a specific panel of microRNAs (miRNA) are used as biomarkers.

The present invention is based in part on the unexpected results obtained when testing the expression level of selected miRNAs in tumor samples obtained from CRC patients and control samples including non-cancerous tissue adjacent to the tumor, lymph nodes and peripheral blood mononuclear cells (PBMC) of healthy individuals. A distinct set of miRNAs was identified, which correlates accurately with the clinical diagnosis of the CRC patients compared to controls. The CRC-specific miRNA panel was found effective in diagnosing and staging CRC with remarkably increased specificity and sensitivity. In particular, the miRNA panel was found to be remarkably effective in diagnosing CRC metastasis, including micrometastatic disease and/or occult metastasis. Moreover, the identified miRNAs could advantageously differentiate between various lymphatic stages of CRC metastasis.

Thus, according to one aspect the present invention provides methods and kits for diagnosing CRC, particularly occult metastasis of CRC, using a plurality of miRNAs comprising miR566, miR96, miR183, miR194, miR200a, miR200b, miR200c, miR203 and miR429. Each possibility is a separate embodiment of the invention. In a particular embodiment, the plurality of miRNAs consists of miR566 and at least one miRNA selected from miR96, miR183, miR194, miR200a, miR200b, miR200c, miR203 and miR429. In another embodiment, the plurality of miRNAs consists of miR194, miR429, miR96, miR183, miR200a, miR200b, miR200c and miR203.

In particular embodiments, the panel of miRNAs comprises hsa-mir-566, hsa-mir-96, hsa-mir-183, hsa-mir-194, hsa-mir-200a, hsa-mir-200b, hsa-mir-200c, hsa-mir-203 and hsa-mir-429. In one specific embodiment the panel of miRNAs consists of hsa-mir-566, hsa-mir-96, hsa-mir-183, hsa-mir-194, hsa-mir-200a, hsa-mir-200b, hsa-mir-200c, hsa-mir-203 and hsa-mir-429.

In one embodiment, hsa-mir566 has the nucleic acid sequence as set forth in SEQ ID NO: 1 (gggcgccugugaucccaac). In another embodiment, hsa-mir-96 has the nucleic acid sequence as set forth in SEQ ID NO: 2 (uuuggcacuagcacauuuuugcu). In another embodiment, hsa-mir-183 has the nucleic acid sequence as set forth in SEQ ID NO: 3 (uauggcacugguagaauucacu). In another embodiment, hsa-mir-194 has the nucleic acid sequence as set forth in SEQ ID NO: 4 (uguaacagcaacuccaugugga). In another embodiment, hsa-mir-200a has the nucleic acid sequence as set forth in SEQ ID NO: 5 (uaacacugucugguaacgaugu). In another embodiment, hsa-mir-200b has the nucleic acid sequence as set forth in SEQ ID NO: 6 (uaauacugccugguaaugauga). In another embodiment, hsa-mir-200c has the nucleic acid sequence as set forth in SEQ ID NO: 7 (uaauacugccggguaaugaugga). In another embodiment, hsa-mir203 has the nucleic acid sequence as set forth in SEQ ID NO: 8 (gugaaauguuuaggaccacuag). In another embodiment, hsa-mir-429 has the nucleic acid sequence as set forth in SEQ ID NO: 9 (uaauacugucugguaaaaccgu). Each possibility is a separate embodiment of the invention.

According to one aspect the present invention provides a method for diagnosing metastasis of CRC in a subject, the method comprising determining the expression level of a plurality of miRNAs selected from the group consisting of miR566, miR96, miR183, miR194, miR200a, miR200b, miR200c, miR203 and miR429, or combinations thereof, in a biological sample obtained from the subject, wherein a significant elevation in the expression levels of the plurality of miRNAs in the biological sample compared to control values indicates that said subject is afflicted with metastasis of CRC.

In one embodiment, said metastasis is micrometastasis. In particular embodiments, the micrometastasis detected using the methods and kits of the present invention has a diameter of less than 10 mm, of less than 8 mm, of less than 6 mm, of less than 5 mm, of less than 4 mm, of less than 3 mm, of less than 2 mm or of less than 1 mm, wherein each possibility is a separate embodiment of the invention. In another embodiment, said micrometastasis has a diameter of less than 0.5 mm. In another embodiment, said micrometastasis has a diameter of about 0.2 mm. In some embodiments, the diameter range of the micrometastasis detectable according to the methods and kits of the present invention are between about 10 mm to about 0.2 mm, between about 8 mm to about 0.2 mm, between about 6 mm to about 0.2 mm, between about 5 mm to about 0.2 mm, between about 4 mm to about 0.2 mm, 3 mm to about 0.2 mm or between about 2 mm to about 0.2 mm. Each possibility is a separate embodiment of the invention.

In another embodiment, said metastasis is sub-micrometastasis. In some embodiments, sub-micrometastasis has a diameter of less than 0.2 mm. In another embodiment, sub-micrometastasis has a diameter of less than 0.1 mm.

In anther embodiment, said metastasis is selected from the group consisting of lymphatic, local and regional metastasis, wherein each possibility is a separate embodiment of the invention. In another embodiment, said metastasis is in the form of circulating tumor cells in the blood, lymphatics or peritoneal cavity. In another embodiment, said metastasis is peritoneal metastasis. In another embodiment, said metastasis is a liver metastasis (e.g., from a colonic origin).

In another embodiment, said metastasis is occult metastasis. In a particular embodiment, said metastasis is occult lymph node metastases. Typically, occult metastasis is a metastasis that is hidden, e.g., not detectable by conventional histopathological methods.

In another embodiment, the method provides detection of minimal residual disease (MRD) of CRC. As used herein “detection of minimal residual disease CRC” or “detection of MRD of CRC” refers to detection of small numbers of cancerous cells (e.g., clusters of 1-10 cells) that remain in the patient during treatment or after treatment when the disease is in remission.

According to another aspect, the present invention provides a method for diagnosing CRC or a precancerous lesion in a subject, the method comprising determining the expression level of a plurality of miRNAs selected from the group consisting of miR566, miR96, miR183, miR194, miR200a, miR200b, miR200c, miR203 and miR429 or combinations thereof, in a biological sample obtained from the subject, wherein a significant elevation in the expression levels of the plurality of miRNAs in the biological sample compared to control values indicates that said subject is afflicted with CRC or a precancerous lesion.

According to another aspect, the present invention provides a method for diagnosing a precancerous lesion in a subject, the method comprising determining the expression level of a plurality of miRNAs selected from the group consisting of miR566, miR96, miR183, miR194, miR200a, miR200b, miR200c, miR203 and miR429 or combinations thereof, in a biological sample obtained from the subject, wherein a significant elevation in the expression levels of the plurality of miRNAs in the biological sample compared to control values indicates that said subject is afflicted with a precancerous lesion.

In a particular embodiment, said precancerous lesion is a premalignant colorectal polyp. In another embodiment, the premalignant colorectal polyp is adenomatous polyp. According to some embodiments, determining the expression levels according to the methods of the invention comprises determining the RNA expression levels of said plurality of miRNAs. Typically, determining RNA expression levels in a sample comprises methods known in the art, such as, amplifying and quantifying said RNA (e.g. using PCR) or hybridization assays (e.g. ISH and FISH).

According to one embodiment, determining the RNA expression levels comprises amplifying RNA extracted from said biological sample. According to another embodiment, amplifying RNA is performed by polymerase chain reaction (PCR). According to another embodiment, the PCR is real-time PCR. According to some embodiments, said PCR is a quantitative real-time PCR (qRT-PCR).

According to another embodiment, determining RNA expression levels is performed using a hybridization assay. According to one embodiment, the hybridization assay is performed using in situ hybridization (ISH). According to one embodiment, the hybridization assay is performed using a solid-phase nucleic acid biochip array.

According to some embodiments, the biological sample is selected from the group consisting of tissue, fluid or excretion samples. In another embodiments, said biological sample is selected from the group consisting of a tissue biopsy, lymph nodes, sentinel lymph nodes, metastatic tissue, blood, serum, plasma, stool, urine and peritoneal wash.

According to another embodiment, the biological sample is an excretion sample selected from urine or stool. According to a particular embodiment, the biological sample is stool. According to some embodiments, the methods of the invention are performed non-invasively.

According to another embodiment, the biological sample is a fluid sample selected from a group consisting of blood, serum, lymph fluid, peritoneal fluid, or lavage of body cavities or organs. According to one embodiment, said biological sample is a blood sample. The fluid sample, in some embodiments, is aspirated from a physiological or pathological fluid. The term may also optionally encompass samples of in vitro cell culture constituents. The sample can optionally be diluted or eluted before performing any other diagnostic assay. Each of the above-described possibilities is a separate embodiment of the present invention.

In one embodiment, said biological sample is a tissue biopsy. In some embodiments, the tissue biopsy is obtained form the colon or rectum of the subject. In certain embodiments the tissue is a fresh, frozen, fixed, wax-embedded or formalin-fixed paraffin-embedded (FFPE) tissue. In particular embodiment, the tissue biopsy is a lymph node biopsy. In yet another particular embodiment, the lymph node is sentinel lymph node (SLN). In another embodiment, there is provided a method for staging CRC in the subject, wherein the biological sample obtained from the subject is a lymph node, the method further comprises determining the number of lymph nodes in said subject expressing said plurality of miRNAs, wherein the number of lymph nodes expressing said plurality of miRNAs indicates the stage of a cancer.

In another embodiment, said method is for monitoring the progression of CRC, recurrence of CRC or monitoring the efficacy of a therapy. In a specific embodiment, said therapy is selected from the group consisting of cytotoxic agents, radiation therapy, biological agents, or immunotherapy.

According to another embodiment, the control value (of each miRNA) correlates to the expression level of said miRNA in a non-cancerous sample (e.g., obtained from the subject being diagnosed or from a healthy subject). Typically, the comparison to the control values is performed in a sample (e.g., tissue, fluid or excretion) specific manner.

According to some embodiments, the control values are determined in a sample obtained from said subject. In one embodiment, the control sample obtained from said subject is an adjacent normal colonic tissue, the control value correlates to the expression level of the miRNA in the adjacent normal colonic tissue.

According to another embodiment, the control values is selected from the group consisting of values obtained from a healthy control individual not afflicted with cancer (e.g., CRC), a panel of control values from a set of healthy individuals not afflicted with cancer, and a stored set of data corresponding to control individuals that are not afflicted with cancer. In some embodiments, said control (non-cancerous) sample is selected from the group consisting of normal colonic tissue obtained from a healthy subject, lymph nodes obtained from a healthy subject or PBMC (e.g. peripheral blood lymphocytes) of a healthy subject.

According to certain embodiments of the methods of the invention, the cancer is invasive cancer. In another embodiment, the cancer is a metastasis of colorectal cancer.

According to another aspect the present invention provides a method for staging colorectal cancer (CRC) in a subject, the method comprising:
(a) obtaining a plurality of lymph node samples from the subject;
(b) determining the number of lymph nodes having a significant elevation in the a expression level of a plurality of miRNAs compared to control values, wherein the plurality of miRNAs is selected from the group consisting of miR96, miR183, miR194, miR200a, miR200b, miR200c, miR203 and miR429, or combination thereof;
   wherein the number of lymph nodes having a significant elevation in the expression level of said plurality of miRNAs indicates the stage of CRC.

In one embodiment, said plurality of miRNAs consists of miR96, miR183, miR194, miR200a, miR200b, miR200c, miR203 and miR429.

According to another aspect, the present invention provides kits suitable for use in diagnosing CRC in a subject, preferably a human. In another embodiment, there is provided a diagnostic kit comprising means for determining the expression level of a plurality of miRNAs selected from the group consisting of miR566, miR96, miR183, miR194, miR200a, miR200b, miR200c, miR203 and miR429. In another embodiment, the kit consists of miR96, miR183, miR194, miR200a, miR200b, miR200c, miR203 and miR429. In another embodiment, the kit consists of miR566, miR96, miR183, miR194, miR200a, miR200b, miR200c, miR203 and miR429.

In anther embodiment, the kit comprises:
(a) miRNA hybridization or amplification reagents; and
(b) at least one probe or amplification primer specific for each member selected from the plurality of miRNAs.

In some embodiments, the kit further comprises means for collecting a sample (e.g., blood, stool) from a subject. In another embodiment, the diagnostic kit further comprises instructions for performing the necessary steps for determining miRNAs expression levels, e.g., in a sample obtained from a subject.

Other objects, features and advantages of the present invention will become clear from the following description and drawings.

### BRIEF DESCRIPTION OF THE DRAWINGS

FIG. 1 shows differentiated expression of 59 miRNA (p<0.01) in a panel of tumor tissue (top left panel, sample designation terminating with A) and adjacent normal tissue (top right panel, sample designation terminating with B) obtained from CRC patients (n=10).

FIG. 2 is a graph showing expression of hsa-mir-96 in human PBMCs obtained from healthy individuals (n=20), pooled normal lymphatic RNA (obtained from Ambion®), paired samples of tumor and adjacent normal (AT) tissues obtained from patients with adenocarcinoma of the colon (n=20), and pooled normal colonic RNA (Ambion®).

FIG. 3 is a graph showing expression of hsa-mir-183 in human PBMCs obtained from healthy individuals (n=20), pooled normal lymphatic RNA (obtained from Ambion®), paired samples of tumor and adjacent normal (AT) tissues obtained from patients with adenocarcinoma of the colon (n=20), and pooled normal colonic RNA (Ambion®).

FIG. 4 is a graph showing expression of hsa-mir-194 in human PBMCs obtained from healthy individuals (n=20), pooled normal lymphatic RNA (obtained from Ambion®), paired samples of tumor and adjacent normal (AT) tissues obtained from patients with adenocarcinoma of the colon (n=20), and pooled normal colonic RNA (Ambion®.

FIG. 5 is a graph showing expression of hsa-mir-200a in human PBMCs obtained from healthy individuals (n=20), pooled normal lymphatic RNA (obtained from Ambion®), paired samples of tumor and adjacent normal (AT) tissues obtained from patients with adenocarcinoma of the colon (n=20), and pooled normal colonic RNA (Ambion®).

FIG. 6 is a graph showing expression of hsa-mir-200b in human PBMCs obtained from healthy individuals (n=20), pooled normal lymphatic RNA (obtained from Ambion®), paired samples of tumor and adjacent normal (AT) tissues obtained from patients with adenocarcinoma of the colon (n=20), and pooled normal colonic RNA (Ambion®).

FIG. 7 is a graph showing expression of hsa-mir-200c in human PBMCs obtained from healthy individuals (n=20), pooled normal lymphatic RNA (obtained from Ambion®), paired samples of tumor and adjacent normal (AT) tissues obtained from patients with adenocarcinoma of the colon (n=20), and pooled normal colonic RNA (Ambion®).

FIG. 8 is a graph showing expression of hsa-mir-203 in human PBMCs obtained from healthy individuals (n=20), pooled normal lymphatic RNA (obtained from Ambion®), paired samples of tumor and adjacent normal (AT) tissues obtained from patients with adenocarcinoma of the colon (n=20), and pooled normal colonic RNA (Ambion®).

FIG. 9 is a graph showing expression of hsa-mir-429 in human PBMCs obtained from healthy individuals (n=20), pooled normal lymphatic RNA (obtained from Ambion®), paired samples of tumor and adjacent normal (AT) tissues obtained from patients with adenocarcinoma of the colon (n=20), and pooled normal colonic RNA (Ambion®).

FIG. 10 shows the differential expression of miR96, miR183, miR194, miR200a, miR200b, miR200c, miR203 and miR429 in tumor tissue compared to normal lymphatic tissue and normal tumor adjacent tissue.

FIG. 11 is a graph showing the expression of miR-183, miR200c, miR203, miR429 and miR96 in PBMCs (n=2), plasma of healthy individuals (n=2, center columns) and plasma of CRC patients (n=2).

FIG. 12 depicts the differential expression of the miRNA panel measured by qPCR in liver metastases.

FIG. 13 depicts the differential expression of the miRNA panel measured by qPCR in peritoneal metastases.

FIG. 14 depicts miR-566 expression in stool and colonic tissue.

FIG. 15 shows the relative expression of the miRNA panel in SLNs of CRC patients.

### DETAILED DESCRIPTION OF THE INVENTION

The present invention provides methods for diagnosing and staging colorectal cancer (CRC), wherein a panel of microRNA selectively expressed in CRC as compared to normal adjacent tissue or lymphocytes of healthy subjects is used as biomarkers. The present invention further provides diagnostic kits comprising reagents suitable for the detection of a panel of miRNAs which are selectively expressed in colorectal cancer for screening and diagnosing primary CRC. The panel of microRNAs comprises a plurality of miRNAs selected from the group consisting of miR566, miR96, miR183, miR194, miR200a, miR200b, miR200c, miR203 and miR429. The methods and kits of the invention are also suitable for follow up after cancer treatment in order to detect disease-recurrence and initiate effective therapy before overt metastasis appears.

As demonstrated hereinbelow, the panel of miRNAs of the invention was particularly selected according to several criteria including: (i) significant up-regulation in colon tumor cells versus normal tumor-adjacent tissue, normal lymphatic tissue and PBMCs, (ii) low expression in normal lymphatic tissue and lymphocytes, and (iii) sufficiently sensitive to identify a small a mount of tumor cells out of a million normal cells). Without wishing to be bound by any theory or mechanism of action, the use of said criteria enabled the selection of a panel of miRNAs specifically useful in diagnosing occult metastasis and minimal residual disease of CRC, with increased specificity and sensitivity. Furthermore, the miRNA panel is advantageously useful in differentiating between various lymphatic stages of CRC subjects.

The use of a panel of miRNAs consisting of miR96, miR183, miR194, miR200a, miR200b, miR200c, miR203, miR429 and miR566, represents an improvement over the state of the art by providing diagnostic assays with high sensitivity and specificity for diagnosing CRC in a subject as well as prognosis, staging and/or monitoring of CRC progression.

According to another embodiment, the present invention provides a method for diagnosing colorectal cancer (CRC) or metastasis of CRC in a subject, the method comprising determining the expression level of a plurality of miRNAs selected from the group consisting of miR566, miR96, miR183, miR194, miR200a, miR200b, miR200c, miR203 and miR429, in a biological sample obtained from the subject, wherein a significant elevation in the expression levels of the plurality of miRNAs in the biological sample compared to control values indicates that said subject is afflicted with CRC metastasis of CRC.

According to another embodiment, the present invention provides a method for diagnosing a precancerous lesion in a subject, the method comprising determining the expression level of a plurality of miRNAs selected from the group consisting of miR566, miR96, miR183, miR194, miR200a, miR200b, miR200c, miR203 and miR429, in a biological sample obtained from the subject, wherein a significant elevation in the expression levels of the plurality of miRNAs in the biological sample compared to control values indicates that said subject is afflicted with a precancerous lesion.

According to another embodiment, the present invention provides a method for staging colorectal cancer (CRC) in a subject, the method comprising:
(a) obtaining a plurality of lymph node samples from the subject;
(b) determining the number of lymph nodes having a significant elevation in the a expression level of a plurality of miRNAs compared to control values, wherein the plurality of miRNAs is selected from the group consisting of miR96, miR183, miR194, miR200a, miR200b, miR200c, miR203 and miR429; wherein the number of lymph nodes having a significant elevation in the expression level of said plurality of miRNAs indicates the stage of CRC.

In another embodiment, the plurality of miRNAs of the methods and kits of the invention comprises the nucleic acid sequences selected from miR566, miR96, miR183, miR194, miR200a, miR200b, miR200c, miR203 and miR429, or sequences at least about 80%, at least about 85%, at least about 90% or at least about 95% identical thereto. Each possibility is a separate embodiment of the invention.

In some embodiments of the invention, the panel of miRNAs comprises hsa-mir-566, hsa-mir-96, hsa-mir-183, hsa-mir-194, hsa-mir-200a, hsa-mir-200b, hsa-mir-200c, hsa-mir-203 and hsa-mir-429. In one specific embodiment the panel of miRNAs consists of hsa-mir-566, hsa-mir-96, hsa-mir-183, hsa-mir-194, hsa-mir-200a, hsa-mir-200b, hsa-mir-200c, hsa-mir-203 and hsa-mir-429. In another specific embodiment the panel of miRNAs consists of hsa-mir-96, hsa-mir-183, hsa-mir-194, hsa-mir-200a, hsa-mir-200b, hsa-mir-200c, hsa-mir-203 and hsa-mir-429.

TABLE 1
Nucleic acid sequences and accession
No. of the microRNAs panel

| | | SEQ | |
|---|---|---|---|
| | | ID | Nucleic acid |
| microRNA | Accession No. | NO: | sequence |
| hsa-miR-566 | MIMAT0003230 | 1 | gggcgccugugaucccaac |
| hsa-miR-96 | MIMAT0000095 | 2 | uuuggcacuagcacauuuu |
| | | | ugcu |
| hsa-miR-183 | MIMAT0000261 | 3 | uauggcacugguagaauuc |
| | | | acu |
| hsa-miR-194 | MIMAT0000460 | 4 | uguaacagcaacuccaugu |
| | | | gga |
| hsa-miR- | MIMAT0000682 | 5 | uaacacugucugguaacga |
| 200a | | | ugu |
| hsa-miR- | MIMAT0000318 | 6 | uaauacugccugguaauga |
| 200b | | | uga |
| hsa-miR- | MIMAT0000617 | 7 | uaauacugccggguaauga |
| 200c | | | ugga |
| hsa-miR-203 | MIMAT0000264 | 8 | gugaaauguuuaggaccac |
| | | | uag |
| hsa-miR-429 | MIMAT0001536 | 9 | uaauacugucugguaaaac |
| | | | cgu |

In another embodiment, a plurality of miRNAs is at least two miRNAs selected from the miRNA panel of the invention. Non limiting examples of at least two miRNAs being indicative for the methods of the invention are miR96 and miR183; or miR200c and miR429. In another embodiment, a plurality of miRNAs is at least three miRNAs selected from the miRNA panel of the invention. A non limiting example of at least three miRNAs being indicative for the methods of the invention is miR96, miR183 and mR194. In another embodiment, a plurality of miRNAs is at least four miRNAs selected from the miRNA panel of the invention. A non limiting example of at least four miRNAs being indicative for the methods of the invention is miR200a, miR200b, miR203 and miR429. In another embodiment, a plurality of miRNAs is at least five miRNAs selected from the miRNA panel of the invention. In another embodiment, a plurality of miRNAs is at least six miRNAs selected from the miRNA panel of the invention. In another embodiment, a plurality of miRNAs is at least seven miRNAs selected from the miRNA panel of the invention. In another embodiment, a plurality of miRNAs is at least eight miRNAs selected from the miRNA panel of the invention.

In some embodiment of the methods of the invention, a significant elevation in the expression level of at least two miRNAs selected form the miRNA panel is indicative of the subject's state (i.e., diagnosis of CRC or a precancerous lesion, CRC metastasis or staging of CRC). In another embodiment, a significant elevation in the expression level of at least three miRNAs selected form the miRNA panel is indicative of the subject's state. In another embodiment, a significant elevation in the expression level of at least four miRNAs selected form the miRNA panel is indicative of the subject's state. In another embodiment, a significant elevation in the expression level of at least five miRNAs selected form the miRNA panel is indicative of the subject's state. In another embodiment, a significant elevation in the expression level of at least six miRNAs selected form the miRNA panel is indicative of the subject's state. In another embodiment, a significant elevation in the expression level of at least seven miRNAs selected form the miRNA panel is indicative of the subject's state. In another embodiment, a significant elevation in the expression level of at least eight miRNAs selected form the miRNA panel is indicative of the subject's state. In another embodiment, a significant elevation in the expression level of miR96, miR183, miR194, miR200a, miR200b, miR200c, miR203 and miR429 is indicative of the subject's state. In another embodiment, a significant elevation in the expression level of miR566, miR96, miR183, miR194, miR200a, miR200b, miR200c, miR203 and miR429 is indicative of the subject's state.

In exemplary embodiments, the methods and diagnostic compositions of the invention are useful in diagnosing primary CRC with invasive characteristics (e.g., that the cancer is associated with micrometastases). The presence of primary cancer with invasive characteristics would suggest that there is likelihood that the patient will develop distant metastatic cancer. The term “distant metastatic cancer” refers to a primary cancer that has spread to areas of the body that are distant to the primary cancer and established secondary cancers.

In some embodiment, the methods and diagnostic compositions of the invention are useful in detecting circulating tumor cells in a subject's blood. The presence of circulating tumor cells in a subject's blood is also indicative of metastasis.

Typically, a patient may be considered to have metastatic cancer or primary cancer with metastatic characteristics when cancer cells have spread to the lymph nodes of the subject. This includes presence of cancer cells in the sentinel lymph nodes, which are the hypothetical first lymph nodes or groups of nodes reached by metastasizing cancer cells from a tumor, and/or other regional lymph nodes. A further example of a pathology where, when present, a patient may be considered to have metastatic cancer is the presence of CRC tumor cells, or tumor lesions with the identical histology as the primary CRC, are present in distant organs (for example, peritoneum, liver, lung, bone, brain and skin).

In one embodiment the methods of the invention further comprise detection of an additional colon cancer marker. In some embodiments, the method of the invention further comprise determining the expression level of CCAT-1 in the biological sample obtained from the subject, wherein a significant elevation in the expression levels of CCAT-1 compared to a control value indicates that said subject is afflicted with CRC. In another embodiment, the kit of the invention further comprises agents suitable for the detection of CCAT1.

International Patent Application No. WO 09/101,620, to an inventor of the present invention, relates to CCAT-1 as a nucleic acid transcript specifically expressed in cancer cells, particularly colon, rectal and lung cancer. WO 09/101,620 provides methods for diagnosing cancer by detecting the expression of CCAT-1 as well as isolated polynucleotides, compositions and kits for use in said diagnostic methods. The contents of WO 09/101,620 are incorporated herein as if set forth in their entirety. In one embodiment, CCAT-1 has the nucleic acid sequence as set forth in SEQ ID NO: 19. Determining CCAT-1 expression level may comprise detection of the expression or expression levels of CCAT-1 polynucleotides via any means known in the art, e.g., amplifying and quantifying CCAT-1 in a sample (e.g. using PCR) or hybridization assays (e.g. ISH and FISH).

In one embodiment the subject is a mammal, preferably a human.

As used herein the term “diagnosing” or “diagnosis” refers to the process of identifying a medical condition or disease by its signs, symptoms, and in particular from the results of various diagnostic procedures, including e.g. detecting the expression of the nucleic acids according to at least some embodiments of the invention in a biological sample obtained from an individual. Furthermore, as used herein the term “diagnosing” or “diagnosis” encompasses screening for a disease, detecting a presence or a severity of a disease, distinguishing a disease from other diseases including those diseases that may feature one or more similar or identical symptoms, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. The diagnostic procedure can be performed in vivo or in vitro.

“Detection” as used herein refers to detecting the presence of a component (e.g., a nucleic acid sequence) in a sample. Detection also means detecting the absence of a component. Detection also means measuring the level of a component, either quantitatively or qualitatively. With respect to the method of the invention, detection also means identifying or diagnosing cancer in a subject. “Early detection” as used herein refers to identifying or diagnosing cancer in a subject at an early stage of the disease (e.g., before the disease causes symptoms).

“Differential expression” as used herein refers to qualitative or quantitative differences in the temporal and/or cellular expression patterns of a transcript within and among cells and tissue. Thus, a differentially expressed transcripts can qualitatively have its expression altered, including an activation or inactivation, in, e.g., normal versus disease tissue. Genes, for instance, may be turned on or turned off in a particular state, relative to another state thus permitting comparison of two or more states. A qualitatively regulated gene or transcript may exhibit an expression pattern within a state or cell type that may be detectable by standard techniques. Some transcripts will be expressed in one state or cell type, but not in both. Alternatively, the difference in expression may be quantitative, e.g., in that expression is modulated, up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

In some embodiments, the term “level” refers to the expression level of a miRNA according to at least some embodiments of the present invention. Typically the level of the miRNA in a biological sample obtained from the subject is different (e.g., increased) from the level of the same miRNA in a similar sample obtained from a healthy individual (examples of biological samples are described herein). Alternatively, the level of the miRNA in a biological sample obtained from the subject is different (e.g., increased) from the level of the same miRNA in a similar sample obtained from the same subject at an earlier time point. Alternatively, the level of the miRNA in a biological sample obtained from the subject is different (e.g., increased) from the level of the same miRNA in a non-cancerous tissue obtained from said subject (e.g., a tumor adjacent tissue). Typically, the expression levels of the miRNA of the invention are independently compared to their respective control level.

The term “expression level” is used broadly to include a genomic expression profile, e.g., an expression profile of miRNAs. Profiles may be generated by any convenient means for determining a level of a nucleic acid sequence e.g. quantitative hybridization of miRNA, labeled miRNA, amplified miRNA, cDNA, etc., quantitative PCR, ELISA for quantitation, and the like, and allow the analysis of differential gene expression between two samples. A subject or tumor sample, e.g., cells or collections thereof, e.g., tissues, is assayed. Samples are collected by any convenient method, as known in the art. According to some embodiments, the term “expression level” means measuring the abundance of the miRNA in the measured samples.

The plurality of miRNAs described herein, optionally includes any sub-combination of markers (i.e., miRNAs), and/or a combination featuring at least one other marker, for example a known marker. As described herein, the plurality of markers is preferably then correlated with cancer. For example, such correlating may optionally comprise determining the concentration of each of the plurality of markers, and individually comparing each marker concentration to a threshold level. Optionally, if the marker concentration is above the threshold level, the marker concentration correlates with cancer. Optionally, a plurality of marker concentrations correlates with cancer. Alternatively, such correlating may optionally comprise determining the concentration of each of the plurality of markers, calculating a single index value based on the concentration of each of the plurality of markers, and comparing the index value to a threshold level. Also alternatively, such correlating may optionally comprise determining a temporal change in at least one of the markers, and wherein the temporal change is used in the correlating step.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers is outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purpose(s).

In the methods of the invention, a “significant elevation” in expression levels of the plurality of miRNAs refers, in different embodiments, to a statistically significant elevation, or in other embodiments to a significant elevation as recognized by a skilled artisan. For example, without limitation, the present invention demonstrates that an increase of about at least two fold, or alternatively of about at least three fold, of the threshold value is associated with CRC.

In additional embodiments, a significant elevation refers to an increase in the expression of a plurality of miRNAs (e.g., at least two, three, four, five, six or seven) selected from the miRNA panel of the invention, compared to the threshold values, such as depicted in Table 5 hereinbelow. In one embodiment, said threshold value for miR96 is about 1.0. In another embodiment, said threshold value for miR183 is about 1.8. In another embodiment, said threshold value for miR194 is about 2.1. In another embodiment, said threshold value for miR200a is about 3.4. In another embodiment, said threshold value for miR200b is about 5.3. In another embodiment, said threshold value for miR200c is about 2.5. In another embodiment, said threshold value for miR203 is about 0.5. In another embodiment, said threshold value for miR429 is about 2.7.

The term “about” as used herein refers to +/−10%.

Diagnostic methods differ in their sensitivity and specificity. The “sensitivity” of a diagnostic assay is the percentage of diseased individuals who test positive (percent of “true positives”). Diseased individuals not detected by the assay are “false negatives”. Subjects who are not diseased and who test negative in the assay are termed “true negatives”. The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In one embodiment, the method distinguishes a disease or condition (particularly cancer) with a sensitivity of at least 70% at a specificity of at least 70% when compared to normal subjects (e.g., a healthy individual not afflicted with cancer). In another embodiment, the method distinguishes a disease or condition with a sensitivity of at least 80% at a specificity of at least 90% when compared to normal subjects. In another embodiment, the method distinguishes a disease or condition with a sensitivity of at least 90% at a specificity of at least 90% when compared to normal subjects. In another embodiment, the method distinguishes a disease or condition with a sensitivity of at least 70% at a specificity of at least 85% when compared to subjects exhibiting symptoms that mimic disease or condition symptoms.

Diagnosis of a disease according to at least some embodiments of the present invention can be affected by determining a level of a polynucleotide according to at least some embodiments of the present invention in a biological sample obtained from the subject, wherein the level determined can be correlated with predisposition to, or presence or absence of the disease (i.e., cancer or a precancerous state).

The term “sample” as used herein means a sample of biological tissue or fluid or an excretion sample that comprises nucleic acids. Such samples include, but are not limited to, tissue or fluid isolated from subjects. Biological samples may also include sections of tissues such as biopsy and autopsy samples, frozen sections, blood, plasma, serum, sputum, stool and mucus. Biological sample also refers to metastatic tissue obtained from, but not limited to, organs such as liver, lung, and peritoneum. Biological samples also include explants and primary and/or transformed cell cultures derived from animal or patient tissues. Biological samples may also be blood, a blood fraction, gastrointestinal secretions, or tissue sample. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

As used herein, a “sample” or “biological sample” refers to a sample of biological tissue, fluid or excretion that comprises nucleic acids (e.g., miRNA). It should be noted that a “biological sample obtained from the subject” may also optionally comprise a sample that has not been physically removed from the subject. In some embodiments the sample obtained from the subject is a body fluid or excretion sample including but not limited to seminal plasma, blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, peritoneal fluid, cyst fluid, lavage of body cavities, broncho alveolar lavage, lavage of the reproductive system and/or lavage of any other organ of the body or system in the body, and stool.

Numerous well known tissue or fluid collection methods can be utilized to collect the biological sample from the subject in order to determine the expression level of the biomarkers of the invention in said sample of said subject.

Examples include, but are not limited to, blood sampling, urine sampling, stool sampling, sputum sampling, aspiration of pleural or peritoneal fluids, fine needle biopsy, needle biopsy, core needle biopsy and surgical biopsy, and lavage. Regardless of the procedure employed, once a biopsy/sample is obtained the level of the biomarkers can be determined and a diagnosis can thus be made. Tissue samples are optionally homogenized by standard techniques e.g. sonication, mechanical disruption or chemical lysis. Tissue section preparation for surgical pathology can be frozen and prepared using standard techniques. In situ hybridization assays on tissue sections are performed in fixed cells and/or tissues.

In a one embodiment, blood is used as the biological sample. If that is the case, the cells comprised therein can be isolated from the blood sample by centrifugation, for example.

As used herein, the terms “nucleic acid” and “polynucleotide” are used interchangeably, and include polymeric forms of nucleotides of any length, either deoxyribonucleotides or ribonucleotides, or analogs thereof. The following are non-limiting examples of polynucleotides: a gene or gene fragment, exons, introns, messenger RNA (mRNA), microRNA transfer RNA (tRNA), ribosomal RNA (rRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The term also includes both double- and single-stranded molecules.

As used herein the term “cDNA” refers to complementary DNA. “cDNA” refers to an isolated polynucleotide, nucleic acid molecule, or any fragment or complement thereof. It may have originated by recombinant techniques or synthetically, be double-stranded or single-stranded, represent coding and/or non-coding 5′ and 3′ sequences.

A gene coding for a miRNA may be transcribed leading to production of a miRNA precursor known as the pri-miRNA. The pri-miRNA may be part of a polycistronic RNA comprising multiple pri-miRNAs. The pri-miRNA may form a hairpin with a stem and loop. The stem may comprise mismatched bases. The hairpin structure of the pri-miRNA may be recognized by Drosha, which is an RNase III endonuclease. Drosha may recognize terminal loops in the pri-miRNA and cleave approximately two helical turns into the stem to produce a 30-200 nt precursor known as the pre-miRNA. Drosha may cleave the pri-miRNA with a staggered cut typical of RNase III endonucleases yielding a pre-miRNA stem loop with a 5′ phosphate and ˜2 nucleotide 3′ overhang. Approximately one helical turn of stem (˜10 nucleotides) extending beyond the Drosha cleavage site may be essential for efficient processing. The pre-miRNA may then be actively transported from the nucleus to the cytoplasm by Ran-GTP and the export receptor Ex-portin-5.

The pre-miRNA may be recognized by Dicer, which is also an RNase III endonuclease. Dicer may recognize the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment.

Although initially present as a double-stranded species, the miRNA may eventually become incorporated as a single-stranded RNA into a ribonucleoprotein complex known as the RNA-induced silencing complex (RISC).

The RISC may identify target nucleic acids based on high levels of complementarity between the miRNA and the mRNA, especially by nucleotides 2-8 of the miRNA. Only one case has been reported in animals where the interaction between the miRNA and its target was along the entire length of the miRNA. This was shown for miR-196 and Hox B8 and it was further shown that miR-196 mediates the cleavage of the Hox B8 mRNA (Yekta et al, Science 2004; 304:594-596). Otherwise, such interactions are known only in plants (Bartel et al., Plant Physiol 2003; 132:709-717).

A number of studies have looked at the base-pairing requirement between miRNA and its mRNA target for achieving efficient inhibition of translation (Bartel, Cell 2004; 116:281-297). In mammalian cells, the first 8 nucleotides of the miRNA may be important (Doench & Sharp, GenesDev 2004; 18:504-511). However, other parts of the microRNA may also participate in mRNA binding. Moreover, sufficient base pairing at the 3′ can compensate for insufficient pairing at the 5′ (Brennecke et al, PloS Biol. 2005; 3:e85). Computation studies, in which miRNA binding on whole genomes is analyzed, have suggested a specific role for bases 2-7 at the 5′ of the miRNA in target binding, but the role of the first nucleotide, found usually to be “A”, was also recognized (Lewis et al, Cell 2005; 120: 15-20). Similarly, nucleotides 1-7 or 2-8 were used by Krek et al., (Nat Genet. 2005; 37:495-500) to identify and validate targets.

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition. miRNAs may direct the RISC to down-regulate gene expression by either of two mechanisms: mRNA cleavage or translational repression. The miRNA may specify cleavage of the mRNA if the mRNA has a certain degree of complementarity to the miRNA. When a miRNA guides cleavage, the cut may be between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

In some embodiments of the methods and kits of the invention, the plurality of miRNAs comprises the nucleic acid sequences selected from pri-miR566, pri-miR96, pri-miR183, pri-miR194, pri-miR200a, pri-miR200b, pri-miR200c, pri-miR203 and pri-miR429, or sequences at least about 80% identical thereto, or fragments thereof. In another embodiments of the methods and kits of the invention, the plurality of miRNAs consists of the nucleic acid sequences selected from pri-miR566, pri-miR96, pri-miR183, pri-miR194, pri-miR200a, pri-miR200b, pri-miR200c, pri-miR203 and pri-miR429, or sequences at least about 80% identical thereto, or fragments thereof.

In another embodiment, pri-miR566 has the nucleic acid as set forth in. SEQ ID NO: 10 (Accession No. MI0003572). In another embodiment, pri-miR96 has the nucleic acid as set forth in SEQ ID NO: 11 (Accession No. MI0000098). In another embodiment, pri-miR183 has the nucleic acid as set forth in SEQ ID NO: 12 (Accession No. MI0000273). In another embodiment, pri-miR194 has the nucleic acid as set forth in SEQ ID NO: 13 (Accession No. MI0000488). In another embodiment, pri-miR200a has the nucleic acid as set forth in SEQ ID NO: 14 (Accession No. MI0000737). In another embodiment, pri-miR200b has the nucleic acid as set forth in SEQ ID NO: 15 (Accession No. MI0000342). In another embodiment, pri-miR200c has the nucleic acid as set forth in SEQ ID NO: 16 (Accession No. MI0000650). In another embodiment, pri-miR203 has the nucleic acid as set forth in SEQ ID NO: 17 (Accession No. MI0000283). In another embodiment, pri-miR429 has the nucleic acid as set forth in SEQ ID NO: 18 (Accession No. MI0001641).

### Hybridization Assays

Detection of a nucleic acid of interest in a biological sample (e.g., miRNA) may optionally be effected by hybridization-based assays using an oligonucleotide probe. Traditional hybridization assays include PCR, reverse-transcriptase PCR, real-time PCR, RNase protection, in-situ hybridization, primer extension, dot or slot blots (RNA), and Northern blots (i.e., for RNA detection). More recently, PNAs have been described (Nielsen et al. 1999, Current Opin. Biotechnol. 10:71-75). Other detection methods include kits containing probes on a dipstick setup and the like.

The term “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using the probe under sufficiently stringent hybridization conditions permits the selective detection of a specific target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support or the like, as sites for hybridization of other oligonucleotides, as restriction enzymes sites or binding sites for other nucleic acid binding enzymes, etc. In certain embodiments, a probe of the invention is included in a nucleic acid that comprises one or more labels (e.g., a reporter dye, a quencher moiety, etc.), such as a 5′-nuclease probe, a FRET probe, a molecular beacon, or the like, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially complementary to one another); stable duplexes may contain mismatched bases or unmatched bases. Modification of the stringent conditions may be necessary to permit a stable hybridization duplex with one or more base pair mismatches or unmatched bases. Sambrook et al., Molecular Cloning: A Laboratory Manual, 3rd Ed., Cold Spring Harbor Laboratory Press, Cold Spring Harbor, N.Y. (2001), which is incorporated by reference, provides guidance for suitable modification. Stability of the target/probe duplex depends on a number of variables including length of the oligonucleotide, base composition and sequence of the oligonucleotide, temperature, and ionic conditions. One of skill in the art will recognize that, in general, the exact complement of a given probe is similarly useful as a probe. One of skill in the art will also recognize that, in certain embodiments, probe nucleic acids can also be used as primer nucleic acids. Exemplary probe nucleic acids include 5′-nuclease probes, molecular beacons, among many others known to persons of skill in the art.

As used herein, “hybridization” refers to a reaction in which at least one polynucleotide reacts to form a complex that is stabilized via hydrogen bonding between the bases of the nucleotide residues. The hydrogen bonding may occur by. Watson-Crick base pairing, in any other sequence-specific manner. A hybridization reaction may constitute a step in a more extensive process, such as the initiation of a PCR reaction.

Hybridization reactions can be performed under conditions of different stringency. Under stringent conditions, nucleic acid molecules at least 60%, 65%, 70%, 75% identical to each other remain hybridized to each other. A non-limiting example of highly stringent hybridization conditions is hybridization in 6*sodium chloride/sodium citrate (SSC) at approximately 45° C., followed by one or more washes in 0.2*SSC and 0.1% SDS at 50° C., at 55° C., or at about 60° C. or more.

When hybridization occurs in an antiparallel configuration between two single-stranded polynucleotides, those polynucleotides are described as complementary.

Hybridization based assays which allow the detection of a biomarker of interest in a biological sample rely on the use of probe(s) which can be 10, 15, 20, or 30 to 100 nucleotides long optionally from 10 to 50, or from 40 to 50 nucleotides long.

Thus, the polynucleotides of the biomarkers of the invention, according to at least some embodiments, are optionally hybridizable with any of the herein described nucleic acid sequences under moderate to stringent hybridization conditions.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides according to at least some embodiments of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others (e.g., Kricka et al. (1992), Academic Press San Diego, Calif.) can be attached to the oligonucleotides. Preferably, detection of the biomarkers of the invention is achieved by using TaqMan assays, preferably by using combined reporter and quencher molecules (Roche Molecular Systems inc.).

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include 3H, 14C, ³²P, P and ³⁵S.

Those skilled in the art will appreciate that wash steps may be employed to wash away excess target polynucleotide or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA).

### Fluorescence In Situ Hybridization (FISH)

An additional NAT test known in the art is Fluorescence In Situ Hybridization (FISH). FISH uses fluorescent single-stranded DNA or RNA probes which are complementary to the nucleotide sequences that are under examination (genes, chromosomes or RNA). These probes hybridize with the complementary nucleotide and allow the identification of the chromosomal location of genomic sequences of DNA or RNA.

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions. Although other primer nucleic acid lengths are optionally utilized, they typically comprise hybridizing regions that range from about 8 to about 100 nucleotides in length. Short primer nucleic acids generally utilize cooler temperatures to form sufficiently stable hybrid complexes with template nucleic acids. A primer nucleic acid that is at least partially complementary to a subsequence of a template nucleic acid is typically sufficient to hybridize with the template for extension to occur. A primer nucleic acid can be labeled (e.g., a SCORPION primer, etc.), if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or otherwise known in the art. One of skill in the art will recognize that, in certain embodiments, primer nucleic acids can also be used as probe nucleic acids.

Amplification of a selected, or target, nucleic acid sequence may be carried out by a number of suitable methods (e.g., Kwoh et al., 1990, Am. Biotechnol. Lab. 8:14). Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides according to at least some embodiments of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one embodiment, RT-PCR is carried out on an RNA sample from a patient under conditions which favor the amplification of the most abundant RNA. In another embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

In particular embodiments, TagMan® microRNA assay may be used for evaluating the expression levels of the microRNAs panel of the invention. Non limiting examples for evaluating the expression level of the microRNAs of the invention are TagMan® microRNA assay ID 001533 for evaluating miR566, assay ID 000186 for evaluating miR96, assay ID 002269 for evaluating miR183, assay ID 000493 for evaluating miR194, assay ID 001502 for evaluating miR200a, assay ID 002251 for evaluating miR200b, assay ID 002300 for evaluating miR200c, assay ID 000507 evaluating miR203, and assay ID 001024 for evaluating miR429.

The nucleic acid (e.g., RNA) for practicing the present invention may be obtained according to well known methods.

Oligonucleotide primers according to at least some embodiments of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The polymerase chain reaction and other nucleic acid amplification reactions are well known in the art. The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

### Polymerase Chain Reaction (PCR)

The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified”.

### Diagnostic Use

According to another aspect, the present invention provides use of means for detecting the levels of a plurality of microRNAs selected from the group consisting of miR96, miR183, miR194, miR200a, miR200b, miR200c, miR203, miR429 and miR566, for the preparation of a diagnostic composition for assessing (or determining) the presence or absence of colorectal cancer (CRC) or a precancerous lesion in a subject. In one embodiment, a significant elevation in the level of the plurality of microRNAs compared to control values indicates that the subject has CRC or a precancerous lesion. In one embodiment, the subject is suspected of having cancer or a precancerous lesion. In another embodiment, the subject is suspected if having CRC or a precancerous lesion.

According to another aspect, the present invention provides a diagnostic composition for use in assessing (or determining) the presence or absence of CRC or a precancerous lesion in a subject, wherein the diagnostic composition comprises means for detecting the levels of a plurality of microRNAs selected from the group consisting of miR96, miR183, miR194, miR200a, miR200b, miR200c, miR203, miR429 and miR566. In one embodiment, a significant elevation in the level of the plurality of microRNAs compared to control values indicates that the subject has CRC or a precancerous lesion.

According to another aspect, the present invention provides a method for diagnosing cancer (e.g., CRC) in a subject in need thereof, the method comprising the steps of:
(a) performing at least one measurement of the expression level of a plurality of miRNAs selected from the group consisting of miR566, miR96, miR183, miR194, miR200a, miR200b, miR200c, miR203 and miR429 in a biological sample obtained from the subject; and
(b) comparing said expression level to a reference expression level of said panel of miRNA in a control sample;
   wherein an increased or significant elevation in the expression level of said plurality of miRNA in the biological sample compared to the control sample indicates a diagnosis of cancer.

The methods of the invention may, in various embodiments, be used as a single diagnostic assay, or in combination with other diagnostic methods such as cytology or cytoscopy, as known in the art.

In at least some embodiments of the present invention, the methods are conducted in situ. In at least some embodiments of the present invention, the methods are conducted on a subject in vivo. Alternatively, the methods of the invention are conducted in vitro. According to some embodiments of the present invention, the methods are conducted with a sample obtained (e.g., isolated) from a subject screened for, having, being predisposed to, suspected of having cancer, particularly colorectal cancer, wherein each possibility is a separate embodiment of the present invention.

The term “cancer” as used herein means to include all types of cancerous growths or oncogenic processes, metastatic tissues or malignantly transformed cells, tissues, or organs, irrespective of histopathologic type or stage of invasiveness.

The term “colorectal cancer” or “CRC” as used herein refers to cancer of the colon, rectum, anus, and/or appendix. The term also encompasses precancerous polyps of the colon.

As used herein, the term “diagnosis” of cancer and/or “diagnosing” cancer encompasses screening for cancer, detecting the presence of or severity of cancer, prognosis of cancer, early diagnosis of cancer, diagnosing a precancerous lesions, staging of cancer, monitoring of cancer progression and/or treatment efficacy and/or relapse of cancer, as well as selecting a therapy and/or a treatment for cancer, optimization of a given therapy for cancer, and/or predicting the suitability of a therapy for specific subjects (e.g., patients) or subpopulations or determining the appropriate dosing of a therapeutic product in patients or subpopulations. Each possibility is a separate embodiment of the present invention.

As used herein the term “staging” of cancer relates to the determination of the cancer stage as achieved for example by analysis of the regional lymph nodes, specifically sentinel lymph nodes.

Features of malignant tumors, distinct from benign tumors, include invasion and metastasis. Malignant tumors are fatal, mostly due to their capacity to invade neighboring tissues and metastasize through the lymphatic system and bloodstream to near by or distant organs. EMT (epithelial-to-mesenchymal transition) has been considered an essential early step to promote tumor metastasis. The most commonly used staging system is the UICC-AJCC TTNM system. AJCC stage is determined by the magnitude of invasion of the primary tumor (T-stage), metastatic spread to regional lymph nodes (N-stage) or to distant organs (M-stage). The survival and prognosis of colorectal cancer patients depends mainly on the disease stage at the time of detection. Global 5-year survival of patients without lyph node involvement (stage 1 and 2) is around 80%. The percentage of survival drops if positive lymph nodes or distant metastasis are detected (stage 3 and 4). Precise determination of the regional lymph nodes status is the most important diagnostic and prognostic factor in surgically resectable colorectal adenocarcinoma that defines the need for adjuvant chemotherapy. Surgical resection is very effective treatment for patients with localized tumors, however approximately 20-25% of patients who were diagnosed as lymph node negative, by conventional histopathologic methods will develop recurrence and will die of disease.

The high rate of recurrence may be attributed to the presence of occult lymph node metastases undetected by conventional histopathology or due to minimal residual disease (MRD) in the form of circulating tumor cells in the blood, lymphatics or peritoneal cavity. Addition of serial sectioning and immunohistochemical cytokeratine analysis (IF-IC) to standard sectioning and H&E staining, improve staging accuracy in 4-39% of patients. RT-PCR technique for tumor molecular markers increases lymphatic staging sensitivity by 15-50%.

In certain embodiments the method of the invention can also be used for the detection of other types of cancer which are shown to selectively express the panel of miRNAs of the invention. In some embodiments, these cancers include pancreatic cancer and cancer of the stomach.

### Diagnostic Kits

According to another aspect, the present invention provides kits suitable for use in diagnosing CRC or a precancerous lesion in a subject, preferably a human. In another embodiment, there is provided a diagnostic kit comprising means for determining the expression level of a plurality of miRNAs selected from the group consisting of miR566, miR96, miR183, miR194, miR200a, miR200b, miR200c, miR203 and miR429, or combinations thereof.

In another embodiment, the plurality of miRNAs consists of miR566, miR96, miR183, miR194, miR200a, miR200b, miR200c, miR203 and miR429. In another embodiment, the plurality of miRNAs consists of miR96, miR183, miR194, miR200a, miR200b, miR200c, miR203 and miR429.

In another embodiment, the kit comprises:
(a) miRNA hybridization or amplification reagents; and
(b) at least one probe or amplification primer specific for each member selected from the plurality of miRNAs.

In some embodiments, the kit further comprises means for collecting a sample (e.g., blood, stool) from a subject. In another embodiment, the diagnostic kit further comprises instructions for performing the necessary steps for determining miRNAs expression levels, e.g., in a sample obtained from a subject.

The following examples are presented in order to more fully illustrate some embodiments of the invention. They should, in no way be construed, however, as limiting the broad scope of the invention.

### EXAMPLES

### Materials and Methods

### Study Design

The study was divided into four main phases:

1. Marker discovery—identification and selection of miRNAs associated with CRC.

2. Characterization of the differential expression profile of the selected miRNAs in colon tumor tissue versus (i) normal adjacent tissue, (ii) normal lymphatic tissue containing normal lymph nodes and (iii) normal peripheral blood mononuclear cells (PBMCs).

3. Detection of occult metastatic disease in sentinel lymph nodes (SLN) of colon cancer patients.

4. Defining the accuracy of the selected miRNA panel in the detection of minimal residual disease (MRD) in CRC patients.

### Patients and Healthy Controls Study Populations

The study protocol was approved by the Independent Ethical Committee (IEC, Helsinki Committee). Patients with histologic diagnosis of adenocarcinoma of the colon (stages 2-3) were offered participation in the study. Patients were included if they met the following inclusion criteria:

1. Age>18 years.

2. No evidence of distant metastasis on cross sectional imaging.

3. Naïve patients with primary colon cancer without prior systemic or radiation therapy.

TABLE 2
Characteristics of study populations
Patients characteristics

| Age | 40-78 years | |
|---|---|---|
| Gender | | |
| Male | 8 | 40% |
| Female | 12 | 60% |
| Location of tumor | | |
| Right Colon | 7 | 35% |
| Left Colon | 7 | 35% |
| Transverse | 1 | 5% |
| Sigmoid | 2 | 10% |
| Rectum | 1 | 5% |
| AJCC (T + N) | | |
| T2N1 | 2 | 10% |
| T3N0 | 7 | 35% |
| T3N1 | 6 | 30% |
| T3N2 | 1 | 5% |
| T3N3 | 1 | 5% |
| T4N0 | 1 | 5% |
| T4N1 | 1 | 5% |
| Positive SLNs (n = 86) | 14 | 16.3% |
| Tumor differentiation | | |
| Moderate/poor | 3 | 15% |
| Moderate | 15 | 75% |
| Moderate/well | 2 | 10% |
| Mucin secretion | 5 | 25% |

Normal lymph node tissue (n=6) and normal peripheral blood lymphocyte samples (n=15) from healthy donors were used as a control. Total human Lymph Node RNA was purchased from Ambion and other RNA samples were collected during surgery from non colon cancer patients (two of these patients were diagnosed with Lymphoma).

### Sample Processing and Total RNA Isolation

One tumor sample, one tumor adjacent tissue sample and all SLN samples of every patient were removed from the liquid nitrogen, weighted and cut to 50-100 mg pieces on dry ice. Total RNA was extracted from the tissues using miRvana, miRNA isolation kit (Ambion, USA), following the manufacturer's instructions. Weighed tissues were comprehensively crushed on dry ice and disrupted with 1 ml per 50-100 mg tissue, denaturizing lysis buffer using a polytron tissue homogenizer for 1 minute on ice. After addition of 100 μl miRNA homogenate additive, the mixture was left to rest on ice for 10 minutes. After the incubation 1 ml Acid-Phenol: Chloroform (PH 4.5, Ambion, USA) was added and the mixture was vortexed for 1 min. Subsequently, the sample was centrifuged for 5 minutes at 10,000×g at room temperature and the upper aqueous phase was transferred to a new tube and the procedure with Acid-Phenol: Chloroform was repeated. The second transferred aqueous phase was mixed by pipetation with 1.25 volumes of room temperature 100% ethanol and was left to rest on ice for 15 minutes. Then the lysate/ethanol mixture was loaded onto the filter cartridge and centrifuged for 15 seconds at 10,000 g at room temperature. The flow-through was discarded and the procedure was repeated until all of the lysate/ethanol mixture was used. Then the cartridge filter was applied with 700 μl miRNA Wash Solution#1 (working solution mixed with ethanol) and centrifuged for 10 seconds at 10,000 g at room temperature. The flow-through was discarded and filter was applied with 500 μl Wash Solution#2/3 (working solution mixed with ethanol) and centrifuged for 10 seconds at 10,000 g at room temperature. The flow-through was discarded and the procedure with 500 μl Wash Solution#2/3 was repeated. In order to remove residual fluid the filter was centrifuged for 1 min at 10,000 g at room temperature, then the cartridge filter was transferred to a new tube and RNA eluted for 1 minute at 13,200 g at room temperature with 100 μl pre-heated (95° C.) DEPC nuclease-free water. The RNA concentration was measured with NanoDrop Spectrophotometer (ND-100, NanoDrop Technologies, USA) and stored at −80° C. until further use.

The whole blood of healthy volunteers (˜10 ml) was centrifuged for 10 minutes at 1,500 g at 4° C. and the collected plasma was replaced with Dulbecco's PBS, without calcium and magnesium solution (Beit Haemek Biological Industries, Israel). Peripheral blood lymphocytes (PBLs) were separated from the whole blood using Ficoll-Paque PLUS (GE Healthcare, Sweden), at 1:1 volume, after centrifugation for 30 minute at 2,700 g at 4° C. Separated plasma was also collected and stored at −70° C.

The collected cells were washed twice in PBS for 10 minutes at 1,500 g at 4° C. (the supernatant was discarded) and mixed by pipetation with denaturizing lysis buffer (miRvana miRNA isolation kit, Ambion, USA). Further RNA extraction from the PBLs continued as described above.

The 1200 μl of collected plasma from healthy volunteers and cancer patients was filtrated (filter size 0.45 μm) and RNA extraction was performed with “miRvana” miRNA isolation kit as described above.

Stool samples were stored at liquid nitrogen. RNA isolation from 150 mg of the stool sample was performed with “miRvana” miRNA isolation kit as described above.

In order to verify the RNA quality, an amount of 1 μg eluted RNA was heated for 15 minutes at 65° C. and electrophoresed in 0.7% agarose gel with a 100 bp leader marker.

### Real Time PCR

The real-time rqPCR of microRNA expression was preformed with TagMan® MicroRNA Assays (Applied Biosystems, USA). In particular, TagMan® microRNA assay ID 001533 was used for evaluating miR566, assay ID 000186 was used for evaluating miR96, assay ID 002269 was used for evaluating miR183, assay ID 000493 was used for evaluating miR194, assay ID 001502 was used for evaluating miR200a, assay ID 002251 was used for evaluating miR200b, assay ID 002300 was used for evaluating miR200c, assay ID 000507 was used for evaluating miR203 and assay ID 001024 was used for evaluating miR429. The RT and real time quantification was carried out on Applied BioSystems 7500 HT Real-Time PCR System.

The synthesis of cDNA was performed using TaqMan MicroRNA assay reverse transcription loop-primers specific for each mature micro RNA of the invention. There was no need to apply DNAse treatment on the RNA samples. The specificity of primers was tested on genomic DNA with real-time probe+ primers TaqMan miRNA assay and no fluorescent signal was detected (data not shown).

The conversion of microRNA from 50 ng of total RNA to cDNA was performed with TagMan® MicroRNA Reverse Transcription Kit (Applied BioSystems, USA) according to manufacturer's instructions. Each 15 μl of RT reaction mix contained from 5 μl of total RNA in concentration 10 ng/μl and kit reagents: 0.15 μl dNTPs (100 mM total), 1.5 μl RT-Buffer ×10, 1 μl RT miRNA specific loop-primer, 0.19 μl RNAse Inhibitor (20 units/μl) and 1μ of Multiscribe reverse transcriptase (50 units/μl). The two-stage reverse transcription incubation profile was: 16° C. for 30 minutes, 42° C. for 30 minutes, 85° C. for 5 minutes, and 4° C. until specimens were further processed. The cDNA was stored at −20° C. until further use.

Real time relative quantitative PCR was performed using real-time PCR miRNA specific primer and FAM-dye fluorescent probe provided with TaqMan MicroRNA Assay. Each reaction mix (20 μl) included 1.3 μl cDNA, 1 μl of primer and mM probe mixture, and 10 μl TagMan® Universal PCR Master Mix, No AmpErase® UNG (Applied BioSystems, USA). The amplification profile was: 50° C. for 2 minutes and 40 cycles: 95° C. for 10 minutes for enzyme activation, 95° C. for 15 seconds for denaturation, 60° C. for 1 minute for anneal/extend—in which fluorescence was acquired. The real-time amplification profile for stool and plasma samples was lengthened to 55 cycles.

Each sample was checked in duplicates and the expression levels of microRNA were normalized to endogenous snoRNU43. The results were analyzed by the 7500 SDS, version 1.2, software (BioSystems, USA).

The expression of microRNA in colonoscopy fluids was normalized to endogenous snoRNU43, endogenous snoRNU44 and endogenous snoRNU6. The results were analyzed by the StepOnePlus software (BioSystems, USA).

### Sentinel Lymph Node Mapping and Histopathologic Examination

Primary cancerous biopsies and their non-cancerous adjacent tissue were collected from 20 patients during standard surgical resection. Nearby tumor draining sentinel lymph nodes (SLNs), 4 nodes on average from each patient (a total of 86 nodes), were mapped after subserosal ex-vivo around tumor injection of 1-2 ml isosulfan blue dye (Lymphazurin 1%; Ben Venue Labs, Bedford, Ohio). Sentinel nodes were defined as the first blue staining nodes to appear within 5-10 minutes of dye injection (as described by, e.g., Stojadinovic and Nissan et al., Annals of Surgery, 2007; 245(6): 846-857). After the blue-stained SLN harvesting, each node was sectioned into two halves. One piece of each node specimen, along with the resected colon and mesentery, were formalin-fixed and submitted for standard pathologic examination (Stojadinovic and Nissan et al., Annals of Surgery, 2007; 245(6): 846-857). After the diagnosis confirmation and tumor staging, according to AJCC guidelines, each tagged sentinel lymph node was paraffin embedded and dissected to four sections, approximately 4 mm thick. All four sections of each paraffin-embedded specimen were examined by routine H&E staining and cytokeratin immunohistochemistry. Cytokeratin immunohistochemistry was done with a pan-specific antibody cocktail (AEI/AE3, CAM5.2, 35bH11; Ventana Medical Systems, Tuscon. AZ). Detailed sentinel lymph node histopathologic evaluation was performed as previously described (Stojadinovic and Nissan et al., Annals of Surgery, 2007; 245(6): 846-857). The remaining halves, together with the collected biopsies, were immediately stored in liquid nitrogen for molecular examination.

### Example 1

### Identification and Selection of CRC-Specific miRNA

Samples from CRC patient were tested on a miRNA microarray (LC sciences, Houston, USA). The samples included RNA obtained from the tumor and from tissue adjacent to the tumor, and two normal colon samples obtained from non cancer patients. The selection criteria were up-regulation of the miRNA expression in most of the patients and a significantly higher expression in tumors versus adjacent tumor tissue.

Other microRNAs, shown to be upregulated in CRC, were selected from a pool of miRNAs (˜900) found in publicly available sources (e.g. WO2009/140670, WO2009/059026, WO2010/004562 and WO2009/111643) and electronic databases (e.g. www.mirbase.org, www.genecards.org).

Candidate miRNAs, from both sources, were checked in-silico for their expression profile in normal lymphatic tissue. Only miRNA which showed very low expression in normal lymphatic tissue and lymphocytes were selected. The selected miRNA were subjected to further validation as outlined below.

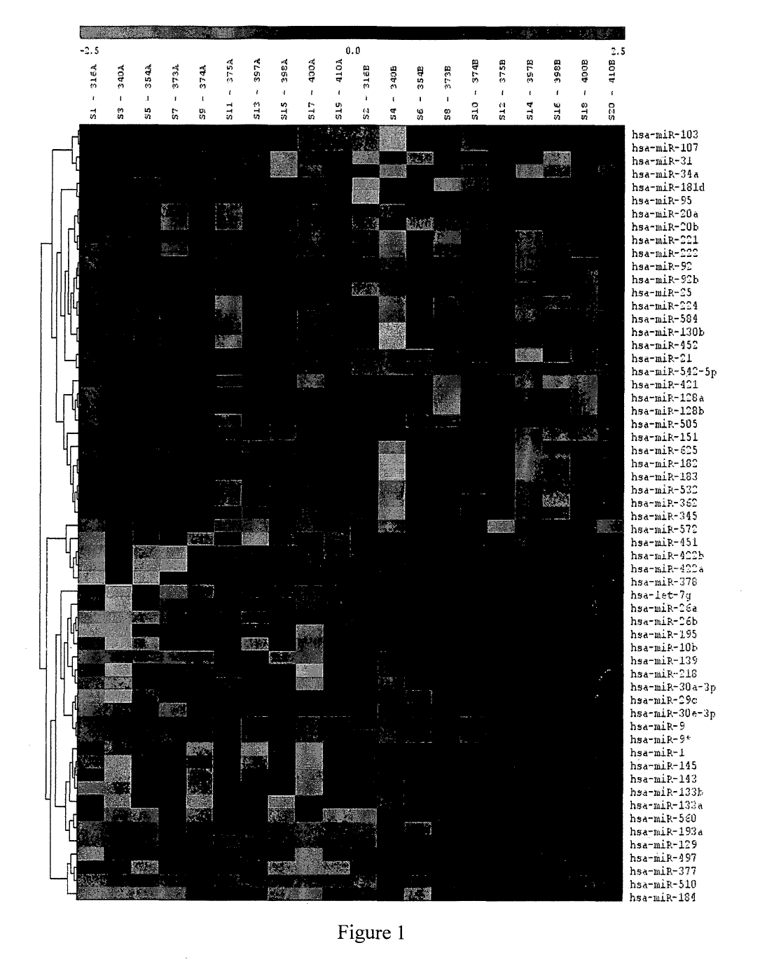

FIG. 1 shows that 59 miRNAs were found to be differentially expressed in tumor tissues as compared to adjacent normal tissues.

### Example 2

### Validation of miRNA Expression in Tumor Tissue, Adjacent Normal Tissue, PBMC and Normal Colon Tissue

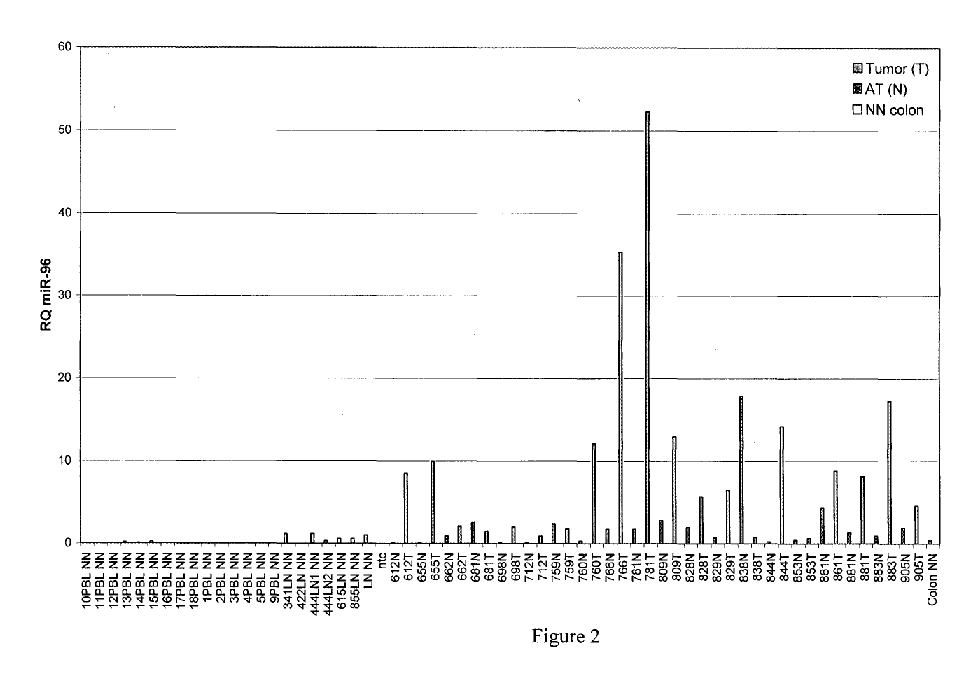

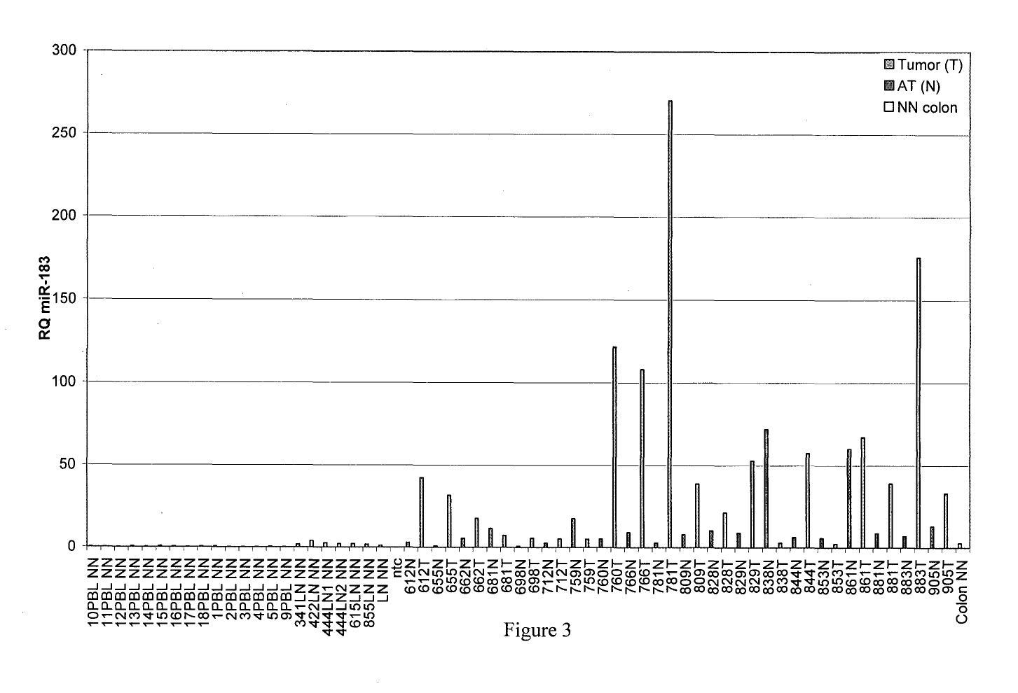

In order to create a diagnostic panel, the expression levels of each miRNA identified (in Example 1) as being over-expressed in colon tumor samples (T) was tested and compared to a panel of (i) paired adjacent normal (AT) tissues obtained from patients with adenocarcinoma of the colon, (ii) PBMCs of healthy individuals (designated PBL) and (iii) RNA extracted from normal colonic tissues (Ambion®) (FIGS. 2-9). Only miRNAs that were exclusively expressed in tumor tissues as validated by real-time PCR were selected for the panel. This selection process resulted in the identification of 8 miRNAs suitable for the diagnostic panel.

FIG. 2 shows the expression profile of hsa-mir-96. Significantly higher expression is seen in tumor samples (T) as compared to the adjacent normal tissue (AT) obtained from patients with adenocarcinoma of the colon, or the PBMC of healthy individuals. Pooled normal colonic RNA (Ambion®) did not show any hsa-mir-96 expression (Right column).

FIG. 3 shows the expression profile of hsa-mir-183. Significantly higher expression is seen in tumor samples as compared to the adjacent normal tissue (AT) obtained from patients with adenocarcinoma of the colon, or the PBMC of healthy individuals. Pooled normal colonic RNA (Ambion®, LN NN Ambion) did not show any hsa-mir-96 expression (Right column).

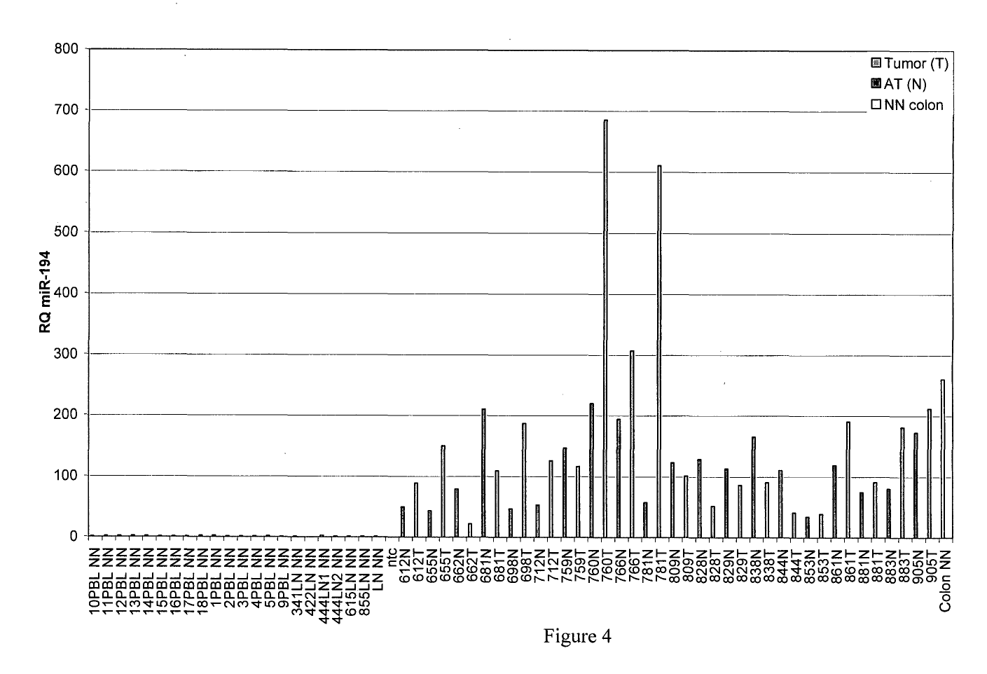

FIG. 4 shows the expression profile of hsa-mir-194. Significantly higher expression is seen in all paired samples of tumor and adjacent normal (AT) tissues from patients with adenocarcinoma of the colon (n=20). hsa-mir-194 was found to be expressed also in pooled normal colonic RNA (Ambion®) (Right column). However, hsa-mir-194 was not expressed in the PBMC of healthy individuals

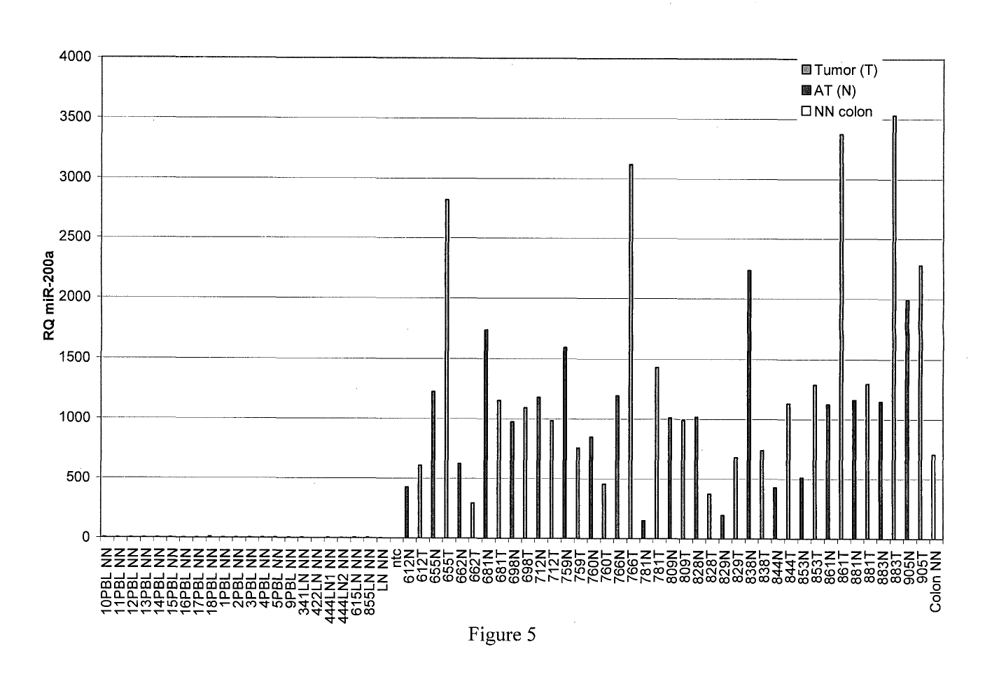

FIG. 5 shows the expression profile of hsa-mir-200a. Significantly higher expression is seen in all paired samples of tumor and adjacent normal (AT) tissues from patients with adenocarcinoma of the colon (n=20). hsa-mir-200a was found to be expressed also in pooled normal colonic RNA (Ambion®) (Right column). However, hsa-mir-200a was not expressed in the PBMC of healthy individuals

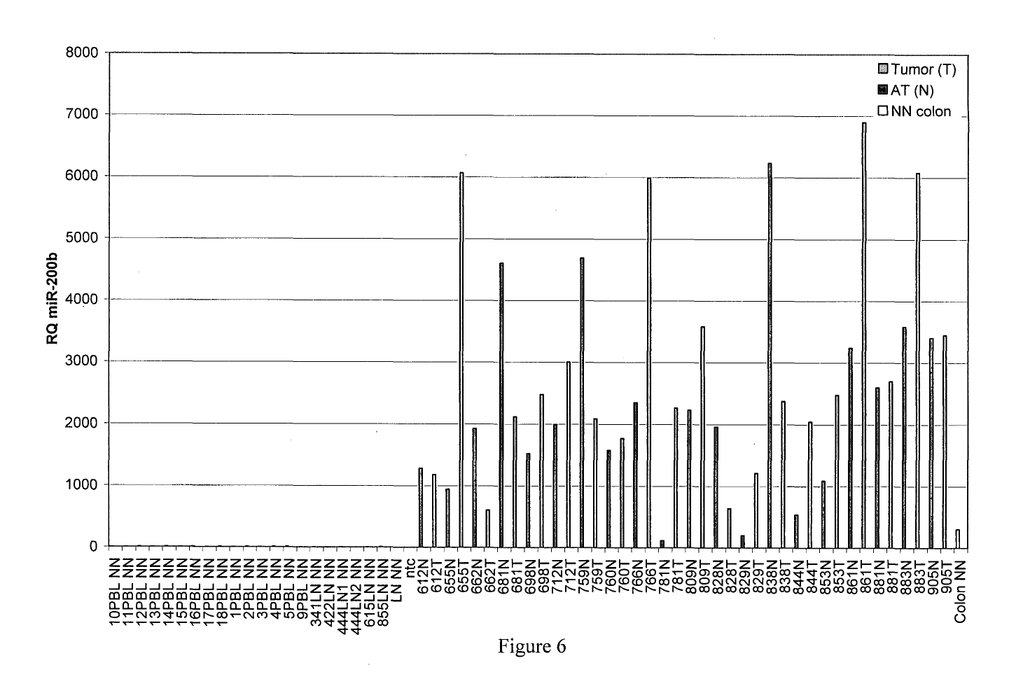

FIG. 6 shows the expression profile of hsa-mir-200b. Significantly higher expression is seen in all paired samples of tumor and adjacent normal (AT) tissues from patients with adenocarcinoma of the colon (n=20). Pooled normal colonic RNA (Ambion®) was show to have low hsa-mir-200b expression (Right column).

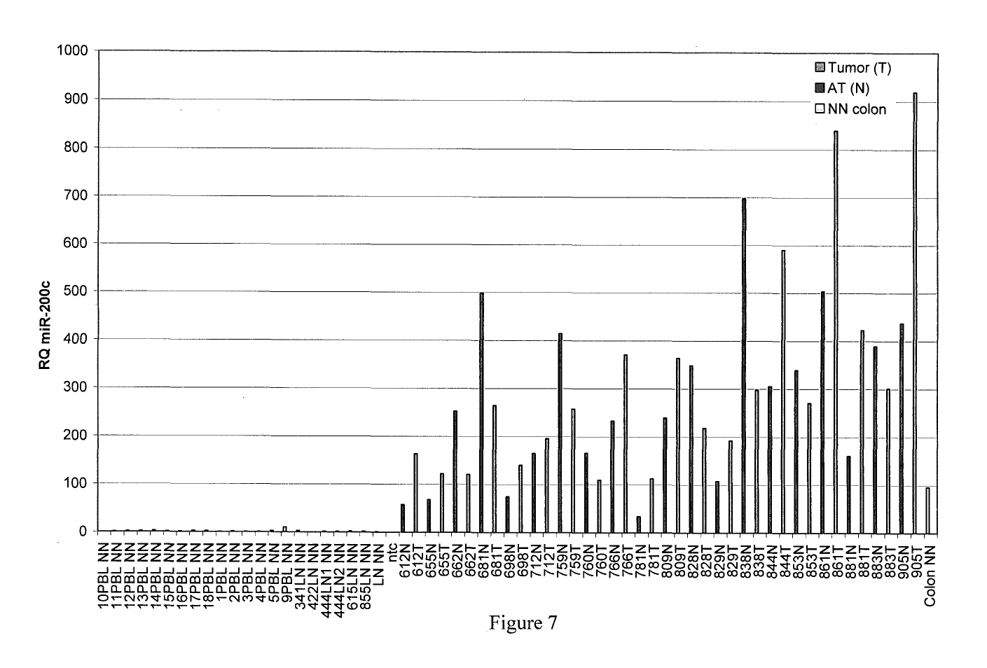

FIG. 7 shows the expression profile of hsa-mir-200c. Significantly higher expression is seen in all paired samples of tumor and adjacent normal (AT) tissues from patients with adenocarcinoma of the colon (n=20). hsa-mir-200c was found to be expressed also in pooled normal colonic RNA (Ambion®) (Right column).

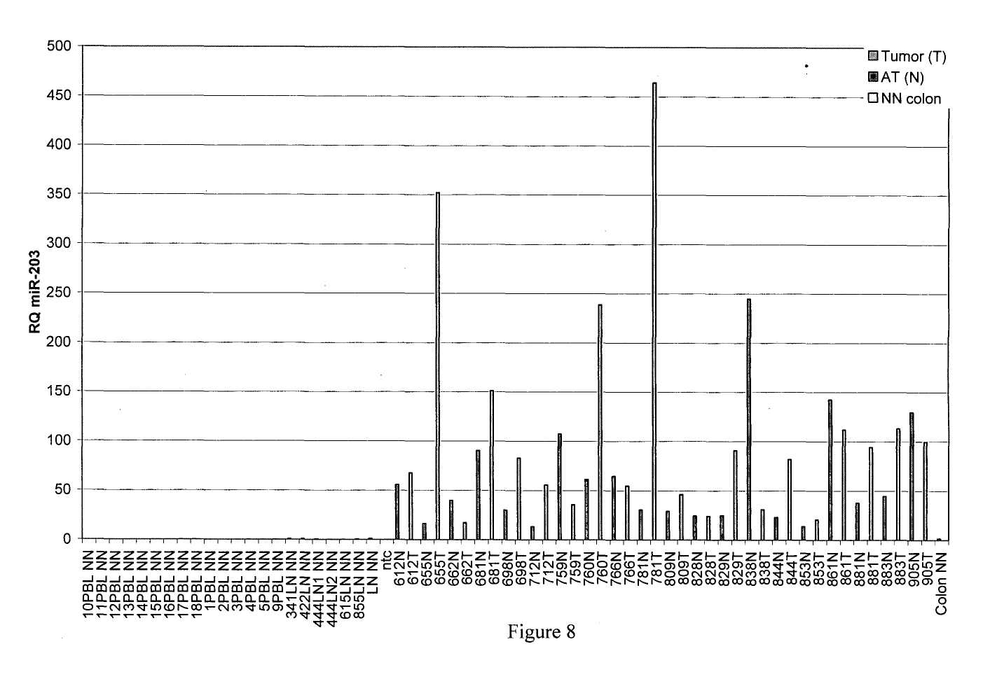

FIG. 8 shows the expression profile of hsa-mir-203. Significantly higher expression is seen in samples of tumor as compared to the paired adjacent normal (AT) tissues obtained from patients with adenocarcinoma of the colon, or the PBMC of healthy individuals. Pooled normal colonic RNA (Ambion®, LN NN Ambion) did not show any hsa-mir-203 expression (Right column).

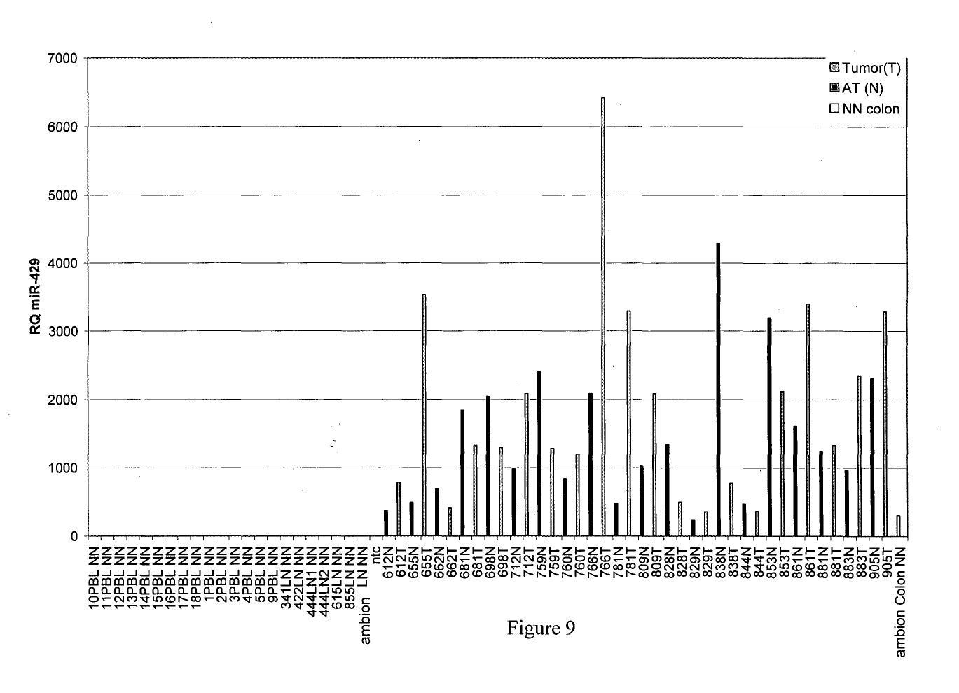

FIG. 9 shows the expression profile of hsa-mir-429. Significantly higher expression is seen in all paired samples of tumor and adjacent normal (AT) tissues from patients with adenocarcinoma of the colon (n=20). Pooled normal colonic RNA (Ambion®) was show to have low hsa-mir-429 expression (Right column).

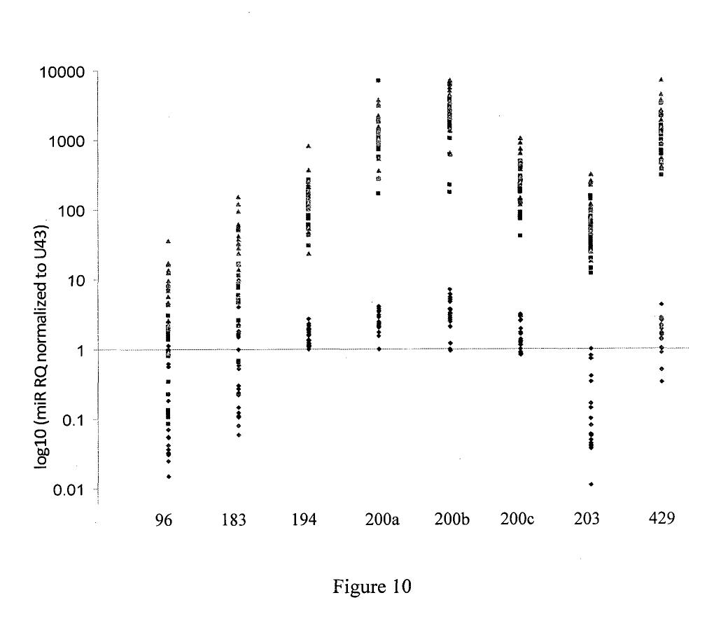

The differential expression of miR96, miR183, miR194, miR200a, miR200b, miR200c, miR203 and miR429 was further demonstrated in FIG. 10 (normal lymphatic tissue depicted as a diamond; normal tumor adjacent tissue depicted as a square and tumor tissue depicted as a triangle).

### Example 3

### Ultrastaging of Sentinel Lymph Nodes of CRC Patients Using the miRNA Panel

In order to assess lymphatic staging, 86 sentinel lymph nodes (SLNs) obtained from 20 CRC patients were studied. Each SLN was cut into two fragments. One half was subjected to enhanced pathological examination using H&E and immunohistochemistry staining for cytokeratin (CK). The other half was snap frozen in liquid nitrogen, RNA was then extracted and the expression of the miRNA panel was studied. Expression of at least two miRNAs from the miRNA panel indicated a CRC positive lymph node. The detailed analysis of SLN ultrastaging of CRC patients is shown in Table 3 below.

TABLE 3
Analysis of SLN ultrastaging of 20 CRC patients

| | | Pathology | | miRNA panel | | | | | | | | Panel |
|---|---|---|---|---|---|---|---|---|---|---|---|---|
| sample | | H&E | CK20 | 96 | 183 | 194 | 200a | 200b | 200c | 203 | 429 | screen |
| 612 | sln1 | neg. | neg. | + | − | − | + | + | + | + | + | + |
| | sln2 | neg. | neg. | − | − | − | − | − | − | − | − | − |
| | sln3 | neg. | neg. | − | − | − | − | − | − | − | − | − |
| | sln4 | pos. | | − | + | + | + | + | + | + | + | + |
| 655 | sln1 | neg. | neg. | + | + | + | + | + | + | + | + | + |
| | sln2 | pos. | | − | − | − | − | − | − | − | − | − |
| | sln3 | neg. | neg. | − | − | − | − | − | − | − | − | − |
| | sln4 | pos. | | + | + | − | − | − | − | + | − | + |
| 662 | sln1 | neg. | pos | + | + | − | − | − | − | − | − | + |
| | sln2 | neg. | pos | − | − | − | − | − | − | − | − | − |
| | sln3 | neg. | pos | − | − | − | − | − | − | − | − | − |
| | sln4 | neg. | pos | − | + | + | − | − | − | − | − | + |
| | sln5 | neg. | pos | + | + | − | − | − | − | − | − | + |
| | sln6 | neg. | pos | − | − | − | − | − | − | − | − | − |
| 681 | sln1 | neg. | neg. | − | − | − | + | + | − | − | + | + |
| | sln2 | neg. | neg. | − | − | − | − | − | − | − | − | − |
| | sln3 | neg. | neg. | − | − | − | + | + | + | + | + | + |
| 698 | sln2 | pos. | | + | + | + | + | + | + | + | + | + |
| | sln3 | neg. | neg. | − | − | − | − | − | − | − | − | − |
| | sln4 | pos. | | − | − | − | − | − | − | − | − | − |
| | sln5 | neg. | neg. | − | − | − | − | − | − | − | − | − |
| 712 | sln1 | neg. | neg. | − | − | − | − | − | − | − | − | − |
| | sln2 | neg. | neg. | − | − | − | − | − | − | − | − | − |
| | sln3 | neg. | neg. | − | − | − | − | − | − | − | − | − |
| | sln4 | neg. | neg. | − | − | − | − | − | − | − | − | − |
| | sln5 | neg. | neg. | − | − | − | − | − | − | − | − | − |
| | sln6 | neg. | neg. | − | − | − | − | − | − | − | − | − |
| 759 | sln1 | neg. | neg. | − | + | − | − | − | − | − | − | − |
| | sln2 | neg. | neg. | − | − | − | − | − | − | − | − | − |
| | sln3 | neg. | neg. | − | − | − | − | − | − | − | − | − |
| 760 | sln1 | pos. | | − | + | + | + | + | + | + | + | + |
| | sln2 | neg. | neg. | − | − | − | − | − | − | − | − | − |
| | sln3 | neg. | neg. | − | − | − | + | + | − | − | + | + |
| 766 | sln1 | neg. | neg. | − | − | − | − | − | − | − | − | − |
| | sln2 | neg. | neg. | − | − | − | − | − | − | − | − | − |
| | sln3 | neg. | neg. | − | + | + | + | + | − | + | + | + |
| 781 | sln1 | neg. | neg. | − | − | − | − | − | − | − | − | − |
| | sln2 | neg. | neg. | − | − | − | − | − | − | − | − | − |
| | sln3 | neg. | neg. | − | − | − | − | − | − | − | − | − |
| | sln4 | neg. | neg. | − | − | − | − | − | − | − | − | − |
| | sln5 | neg. | neg. | − | − | − | − | − | − | − | − | − |
| | sln6 | neg. | neg. | − | − | − | − | − | − | − | − | − |
| | sln7 | neg. | neg. | − | − | − | − | − | − | − | − | − |
| 809 | sln1 | neg. | neg. | − | − | − | − | − | − | − | − | − |
| | sln2 | neg. | neg. | − | + | − | + | + | − | − | + | + |
| | sln3 | neg. | neg. | − | − | − | − | − | − | − | − | − |
| | sln4 | neg. | neg. | − | − | − | − | − | − | − | − | − |
| | sln5 | neg. | neg. | − | + | + | + | + | + | + | + | + |
| 828 | sln1 | neg. | neg. | + | + | − | + | + | − | + | + | + |
| | sln2 | neg. | neg. | − | − | − | − | − | − | − | − | − |
| | sln3 | neg. | neg. | − | − | − | − | − | − | − | − | − |
| | sln4 | neg. | neg. | − | − | − | − | − | − | − | − | − |
| 829 | sln1 | adipose | N/A | N/A | N/A | N/A | N/A | N/A | N/A | N/A | N/A | N/A |
| | sln2 | pos. | | + | + | + | + | + | + | + | + | + |
| | sln3 | neg. | neg. | + | + | + | + | + | + | + | + | + |
| 838 | sln1 | neg. | neg. | − | − | − | − | − | − | − | − | − |
| | sln2 | neg. | neg. | − | − | − | − | − | − | − | − | − |
| | sln3 | neg. | neg. | − | − | − | − | − | − | − | − | − |
| | sln4 | neg. | neg. | − | − | − | − | − | − | − | − | − |
| | sln5 | neg. | neg. | − | − | − | − | − | − | − | − | − |
| 844 | sln1 | neg. | neg. | − | − | − | − | − | − | − | − | − |
| | sln2 | neg. | neg. | − | − | − | − | − | − | − | − | − |
| | sln3 | neg. | neg. | − | − | − | − | − | − | − | − | − |
| | sln4 | adipose | N/A | N/A | N/A | N/A | N/A | N/A | N/A | N/A | N/A | N/A |
| 853 | sln1 | neg. | neg. | − | − | − | − | − | − | − | − | − |
| | sln2 | neg. | neg. | − | − | − | − | − | − | − | − | − |
| | sln3 | neg. | neg. | − | − | − | − | − | − | − | − | − |
| | sln4 | neg. | neg. | − | − | − | − | − | − | − | − | − |
| | sln5 | pos. | | − | − | − | − | − | − | − | − | − |
| 861 | sln1 | neg. | neg. | − | − | − | − | − | − | − | − | − |
| | sln2 | neg. | neg. | − | − | − | − | − | − | − | − | − |
| | sln3 | pos. | | − | − | + | + | + | + | − | + | + |
| | sln4 | neg. | neg. | − | − | − | − | − | − | − | − | − |
| | sln5 | | ? | − | − | − | − | − | − | − | − | − |
| 881 | sln2 | neg. | neg. | − | − | − | − | − | − | − | − | − |
| | sln3 | pos. | | − | + | + | + | + | + | + | + | + |
| | sln4 | pos. | | + | + | + | + | + | + | + | + | + |
| | sln5 | pos. | | − | − | + | + | + | + | − | + | + |
| 883 | sln1 | neg. | neg. | − | − | − | − | − | − | − | − | − |
| | sln2 | neg. | neg. | − | − | − | − | − | − | − | − | − |
| | sln3 | neg. | neg. | − | − | − | − | − | − | − | − | − |
| 905 | sln1 | neg. | neg. | − | − | − | − | − | − | − | − | − |
| | sln2 | neg. | neg. | − | − | − | − | − | − | − | − | − |
| | sln3 | neg. | neg. | − | − | − | − | − | − | − | − | − |
| | sln4 | neg. | neg. | − | − | − | − | − | − | − | − | − |
| | sln5 | neg. | neg. | + | + | + | − | − | + | + | − | + |
| | sln6 | neg. | neg. | − | − | − | − | − | − | − | − | − |

The overall sensitivity of lymphatic staging was increased from 14% as measured by H&E and 21% by immunohistochemistry to 25% by molecular analysis using the miRNA panel.

### Example 4

### Detection of Colorectal Cancer Using the miRNA Panel in Blood Samples

The detection of CRC in blood samples may be performed by identifying the expression levels of the miRNA panel in circulating tumor cells (CTC), or by identification of CRC-specific miRNAs which are cell-free in the plasma or serum.

In order to study the sensitivity of each of the individual miRNAs in the panel for the detection of CTC in a population of normal cells, a calibration curve was constructed using a mixture of PBMCs obtained from healthy individuals mixed with increasing concentrations of the colon cancer cell line HT29.

### Calibration Plot and HT-29 CRC Cell Line Treatment

HT-29 CRC cells were grown in RPMI-1640 medium with 10% FBS, 1% L-Glutamin, 1% PenStrep, 1% Sodium-Pyrovate and 1% HEPES (Beit Haemek Biological Industries, Israel). After removal of the growth medium, the cells were incubated with 2 ml Trypsin-EDTA 0.25% solution B (Beit Haemek Biological Industries, Israel) for 2 minutes in room temperature and washed with Dulbecco's PBS, without calcium and magnesium solution (Beit Haemek Biological Industries, Israel). The cells were counted and diluted with Dulbecco's PBS to sequential concentrations and were mixed with 10⁶ PBMCs of a healthy subject (processed as described above).

After centrifugation at 1500 g for 10 minutes at 4° C. and removal of PBS, RNA was extracted from every cell mixture and every sample was subjected to real-time PCR validation of miRNA panel expression. The higher dilutions of 1:1-1:100 were performed in monoplicates, 1:200-1:4000 in duplicates, 1:10⁴-1:10⁵ in triplicates.

TABLE 4
Calibration curve for miRNA panel

| | miR- | miR- | miR- | miR- | miR- | miR- | miR- |
|---|---|---|---|---|---|---|---|
| | 183 | 194 | 200a | 200b | 200c | 203 | 429 |
| LY 10P\hat{ }6 | 1 | 1 | 1 | 1 | 1 | 1 | 1 |
| HT29 10\hat{ }6 | 1.16E+03 | 144.408 | 5.68E+03 | 2.67E+03 | 294.961 | 1.19E+03 | 5.23E+03 |
| (HT29:LY) | 1.33E+03 | 136.109 | 3.72E+03 | 2.08E+03 | 233.683 | 1.03E+03 | 5.92E+03 |
| 1:1 | | | | | | | |
| 1:2 | 1.28E+03 | 121.41 | 3.52E+03 | 1.69E+03 | 177.151 | 919.219 | 4.74E+03 |
| 1:10 | 311.258 | 29.059 | 791.126 | 97.315 | 66.783 | 217.509 | 1.03E+03 |
| 1:20 | 186.555 | 10.866 | 309.361 | 274.02 | 38.882 | 159.903 | 894.202 |
| 1:100 | 32.231 | 2.73 | 55.463 | 40.481 | 4.904 | 30.681 | 134.317 |
| 1:200 | 16.697 | 1.438 | 20.371 | 15.031 | 2.649 | 8.256 | 39.601 |
| 1:1000 | 2.307 | 1.114 | 4.896 | 4.202 | 0.763 | 2.202 | 10.61 |
| 1:2000 | 0.52 | 0.622 | 1.609 | 1.131 | 0.724 | 0.739 | 2.973 |
| 1:4000 | 0.508 | 0.851 | 1.445 | 1.043 | 0.811 | 1.357 | 2.464 |
| 1:10000 | 0.586 | 0.646 | 1.171 | 0.696 | 0.757 | 0.69 | 1.063 |
| 1:20000 | 0.748 | 0.905 | 1.088 | 1.048 | 1.089 | 0.776 | 1.677 |
| 1:100000 | 0.455 | 0.824 | 1.263 | 1.454 | 1.042 | 1.828 | 2.425 |

A good correlation was found between the presence of the HT-29 (CRC) cell line and miRNA expression. Threshold concentration for CRC detection ranged from 1:20,000 for hsa-mir-200a to 1:200 for hsa-mir-200c. The specific threshold for each miRNA is depicted in bold in the above table 4.

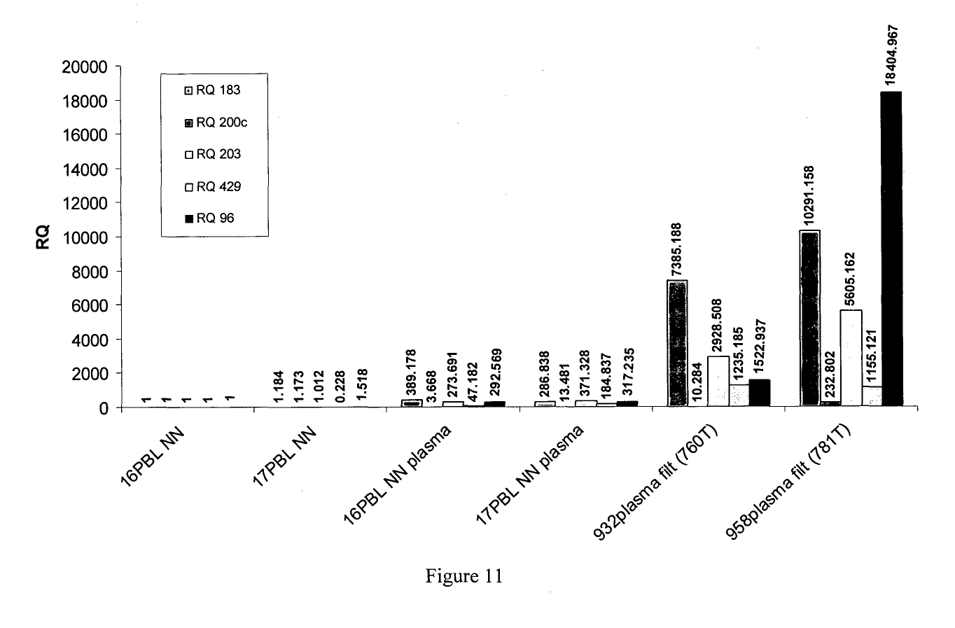

RNA was extracted from plasma and PBMCs of healthy individuals (n=2; 16PBL and 17PBL) and CRC patients (n=2; left columns). There was a significant increase in miRNA panel expression in the plasma of CRC patients compared to normal controls (FIG. 11).

### Example 5

### Threshold Determination for Minimal Residual Disease (MRD) Detection

The threshold value of each miRNA, useful for MRD detection, was defined as the sum of the mean RQ and the standard deviation (stdev) in normal controls.

TABLE 5
Threshold determination for MRD detection

| | miR- | miR- | miR- | miR- | miR- | miR- | miR- | miR- |
|---|---|---|---|---|---|---|---|---|
| | 96 | 183 | 194 | 200a | 200b | 200c | 203 | 429 |
| avg all cont. | 0.39 | 0.77 | 1.64 | 2.57 | 3.56 | 1.70 | 0.21 | 1.72 |
| stdev all cont. | 0.62 | 1.03 | 0.46 | 0.85 | 1.77 | 0.76 | 0.28 | 0.92 |
| Threshold | 1.0 | 1.8 | 2.1 | 3.4 | 5.3 | 2.5 | 0.5 | 2.7 |
| 10PBL NN | 0.015 | 0.08 | 1.345 | 2.442 | 2.831 | N/A | 0.012 | 0.325 |
| 11PBL NN | 0.037 | 0.107 | 1.622 | 2.444 | 2.49 | 1.127 | 0.071 | 1.338 |
| 12PBL NN | 0.183 | 0.529 | 1.957 | 2.384 | 7.132 | 1.631 | 0.062 | 2.179 |
| 13PBL NN | 0.07 | 0.239 | 2.147 | 2.252 | 3.753 | 2.549 | 0.049 | 1.92 |
| 14PBL NN | 0.227 | 0.59 | 1.864 | 2.008 | 6.179 | 2.979 | 0.043 | 4.242 |
| 15PBL NN | 0.055 | 0.272 | 1.3 | 1.538 | 2.602 | 1.358 | 0.067 | 1.354 |
| 16PBL NN | 0.031 | 0.123 | 1.206 | 2.895 | 4.733 | 1.239 | 0.061 | 1.601 |
| 17PBL NN | 0.015 | 0.298 | 1.199 | 2.109 | 2.052 | 3.137 | 0.179 | 2.602 |
| 1PBL NN | 0.033 | 0.222 | 2.684 | 2.064 | 3.225 | 0.848 | 0.058 | 1.019 |
| 2PBL NN | 0.042 | 0.059 | 1.882 | 3.548 | 5.174 | 1.63 | 0.036 | 0.491 |
| 3PBL NN | 0.054 | 0.11 | 1.754 | 2.149 | 3.235 | 0.888 | 0.036 | 0.863 |
| 4PBL NN | 0.032 | 0.106 | 1.988 | 3.829 | 4.849 | 0.814 | 0.095 | 2.496 |
| 5PBL NN | N/A | 0.227 | 2.284 | 4.088 | 5.478 | 2.896 | 0.044 | 2.08 |
| 9PBL NN | 0.025 | 0.148 | 1.092 | 2.107 | 3.675 | 1.287 | 0.09 | 1.357 |
| 341LN NN | 1.124 | 1.496 | 1.129 | 2.236 | 2.13 | 2.52 | 0.692 | 1.572 |
| 422LN NN | 2.446 | 4.067 | 1.759 | 3.406 | 0.95 | 1.646 | 0.708 | 2.758 |
| 444LN NN | 0.827 | 2.249 | 1.849 | 1.755 | 1.203 | 1.124 | 0.235 | 1.022 |
| 615LN NN | 0.563 | 1.805 | 1.115 | 3.061 | 3.022 | 1.942 | 0.15 | 1.671 |
| 855LN NN | 0.62 | 1.702 | 1.561 | 4.096 | 5.549 | 1.712 | 0.423 | 2.572 |
| LN NN ambion | 1 | 1 | 1 | 1 | 1 | 1 | 1 | 1 |

### Example 6

### Performance of the microRNAs Panel in Detection of Tumor Tissue

The performance of the microRNAs panel and the added value of the microRNAs in detection of tumor tissue versus normal controls including lymphatic tissues and normal colonic tissues are shown in Table 6. The results show high sensitivity and specificity in differentiating tumor form normal tissues. These findings support the ability of such microRNA panel to be used for the detection of cancer cells in the blood, stool samples and in lymph nodes

TABLE 6
Performance of the microRNAs panel in detection of tumor tissue

| | | miR- | miR- | miR- | miR- | miR- | miR- | miR- | miR- |
|---|---|---|---|---|---|---|---|---|---|
| | | 96 | 183 | 194 | 200a | 200b | 200c | 203 | 429 |
| Tumor vs. | Sensitivity | 100% | 90% | 75% | 75% | 75% | 75% | 75% | 75% |
| tumor | Specificity | 100% | 95% | 75% | 80% | 80% | 75% | 85% | 75% |
| adjacent | | | | | | | | | |
| tissue (TA) | | | | | | | | | |
| Tumor vs. | Sensitivity | 75% | 100% | 100% | 100% | 100% | 100% | 100% | 100% |
| normal | Specificity | 85% | 100% | 100% | 100% | 100% | 100% | 100% | 100% |
| lymphatic | PPV | 90% | 100% | 100% | 100% | 100% | 100% | 100% | 100% |
| tissue | NPV | 90% | 100% | 100% | 100% | 100% | 100% | 100% | 100% |
| (NL) | | | | | | | | | |
| Tumor | Mean RQ | 8.88 | 43.94 | 189.51 | 1704.88 | 3769.95 | 385.88 | 113.04 | 2309.80 |
| TA | Mean RQ | 1.204 | 8.14 | 115.47 | 1253.12 | 2115.04 | 257.87 | 49.82 | 1270.24 |

The p-value for Tumor vs. tumor adjacent tissue (TA) was as follows: miR-96 p=0.0002; miR183 p=0.0004; miR194 p=0.037; miR200a p=0.127; miR200b p=0.003; miR200c p=0.033; miR203 p=0.002; and miR429 p=0.009. The p-value for tumor vs. normal lymphatic tissue (NL) was p<0.0001.

### Example 7

### Relative Expression of the microRNA Panel in Sentinel Lymph Nodes of CRC Patients

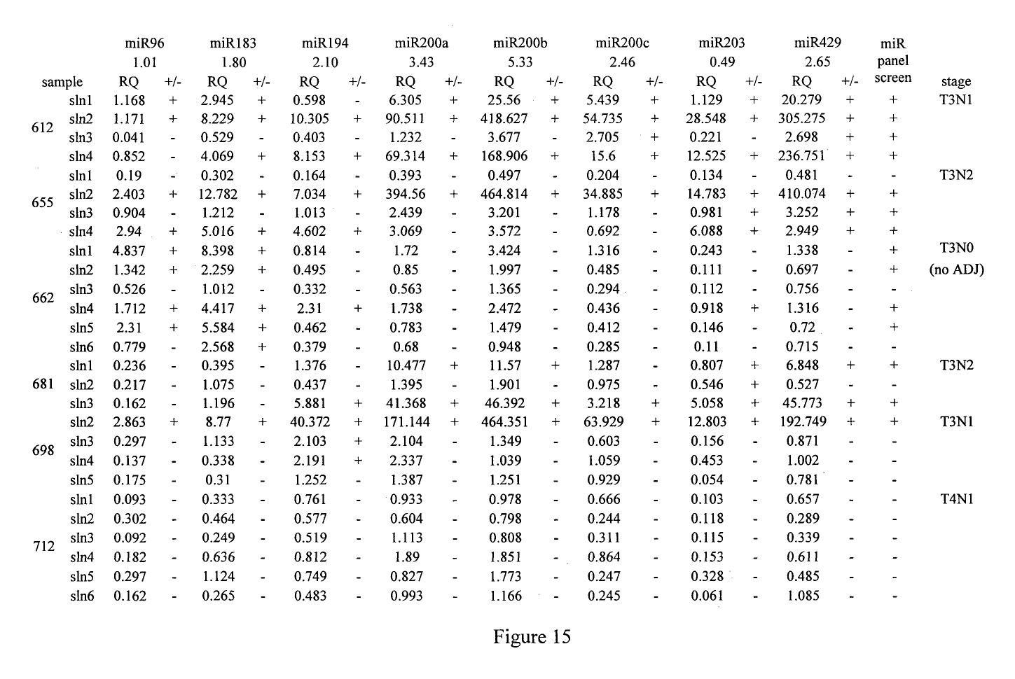

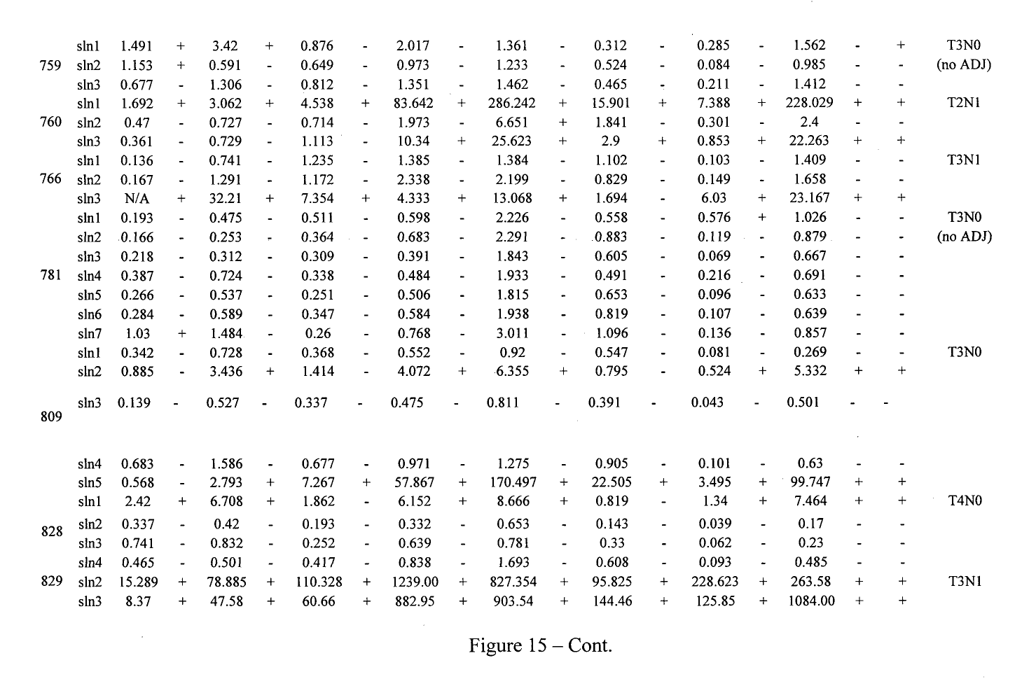

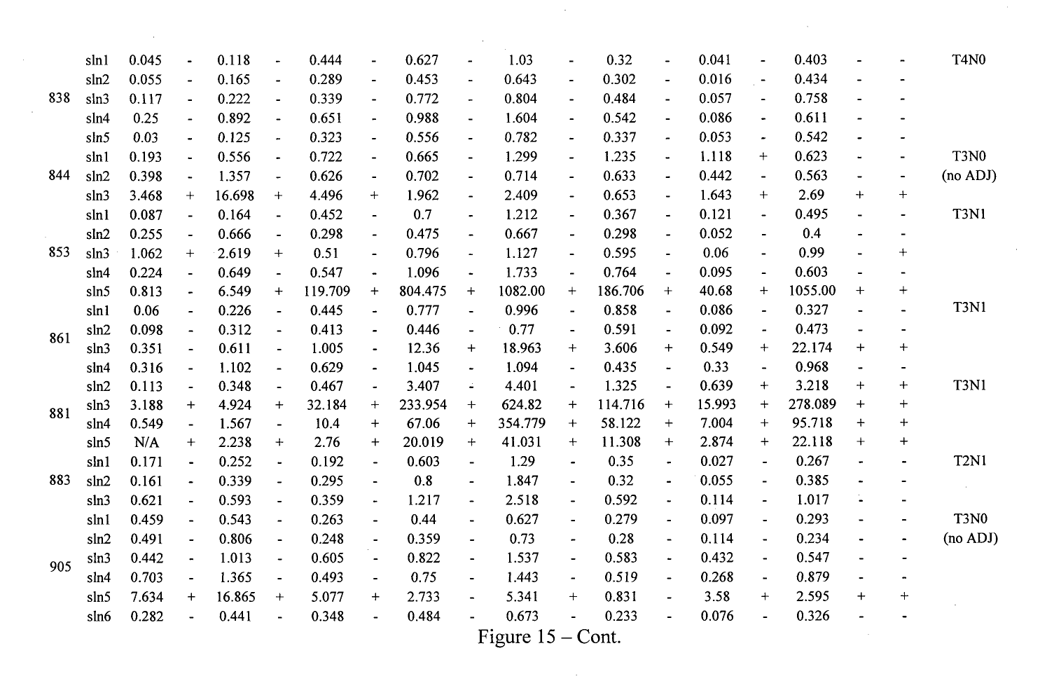

Lymphatic ultrastaging of 86 SLNs was examined using the threshold value determined for minimal residual disease (MRD) detection of CRC patients (as described in Example 5 above). FIG. 15 shows a detailed analysis of SLN ultrastaging of CRC patients, and indicates that MRD can be detected, e.g., in SLN of CRC patients, using the threshold value specific for each miRNA.

Further, the performance of the microRNA panel in the detection of occult metastasis in sentinel lymph nodes of colon cancer patients was calculated. It is important to note that false positivity may be a reflection of greater sensitivity for the detection of occult metastatic disease and not a false result. These results reveal that the miRNA panel may be used as an assay for diagnosing MRD of CRC with high sensitivity and specificity (as shown in Table 7 below). In particular, the sensitivity of lymph node metastasis detection using H&C was about 14%; using H&C and CK was about 21%; using the miRNA panel was about 38%; and using H&E, CK and the miRNA panel was over 40%.

TABLE 7
Performance of the microRNA panel in detection of occult metastasis

| number of | miR- | miR- | miR- | miR- | miR- | miR- | miR- | miR- |
|---|---|---|---|---|---|---|---|---|
| SLNs | 96 | 183 | 194 | 200a | 200b | 200c | 203 | 429 |
| TP | 11 | 14 | 12 | 10 | 10 | 10 | 12 | 11 |
| FP | 11 | 11 | 8 | 10 | 12 | 7 | 17 | 15 |
| FN | 7 | 4 | 6 | 8 | 8 | 8 | 6 | 7 |
| TN | 55 | 55 | 58 | 56 | 54 | 59 | 49 | 51 |
| Sensitivity | 61% | 78% | 67% | 56% | 56% | 56% | 67% | 61% |
| Specificity | 83% | 83% | 88% | 85% | 82% | 89% | 74% | 77% |
| PPV | 50% | 56% | 60% | 50% | 45% | 59% | 41% | 42% |
| NPV | 89% | 93% | 91% | 88% | 87% | 88% | 89% | 88% |
| Accuracy | 79% | 82% | 83% | 79% | 76% | 82% | 73% | 74% |

| | |
|---|---|
| Panel | 83% |
| sensitivity | |
| Panel | 74% |
| specificity | |
| Panel PPV | 47% |
| Panel NPV | 94% |
| Panel Accuracy | 76% |

### Example 8

### Relative Expression of the microRNAs Panel in Distant Metastases from Colonic Origin

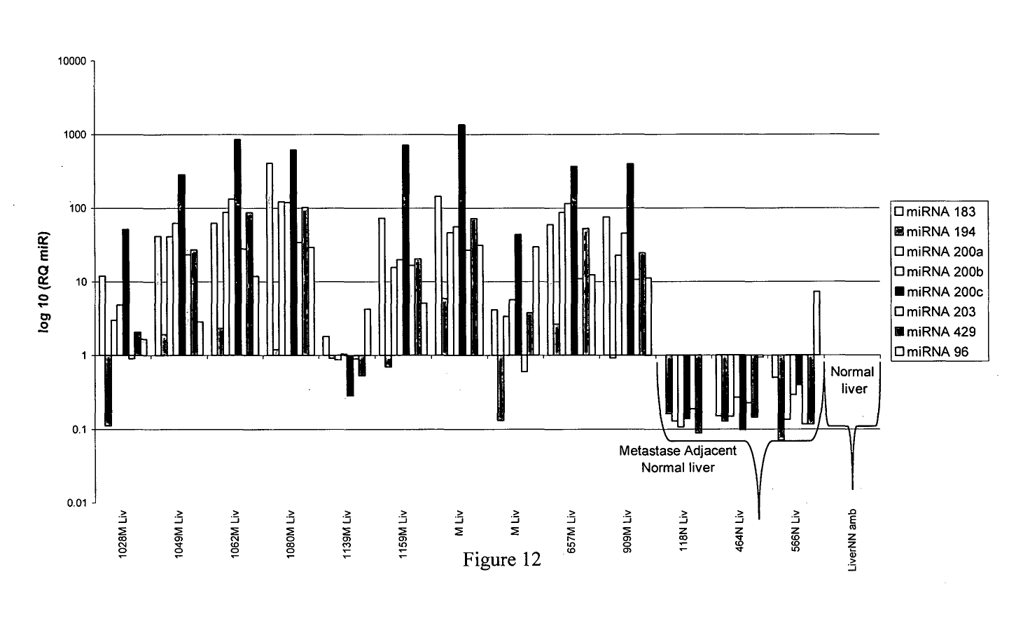

The potential of the microRNA panel to serve for the detection of distant metastasis such as liver metastasis was evaluated. qPCR assays were performed for the 8 microRNA fragments in human tissues obtained from patients operated on for liver metastasis originating from colon cancer. The relative expression of the 8 selected microRNA was high in 9/12 (75%) of the liver metastases compared to normal liver tissue (FIG. 12).

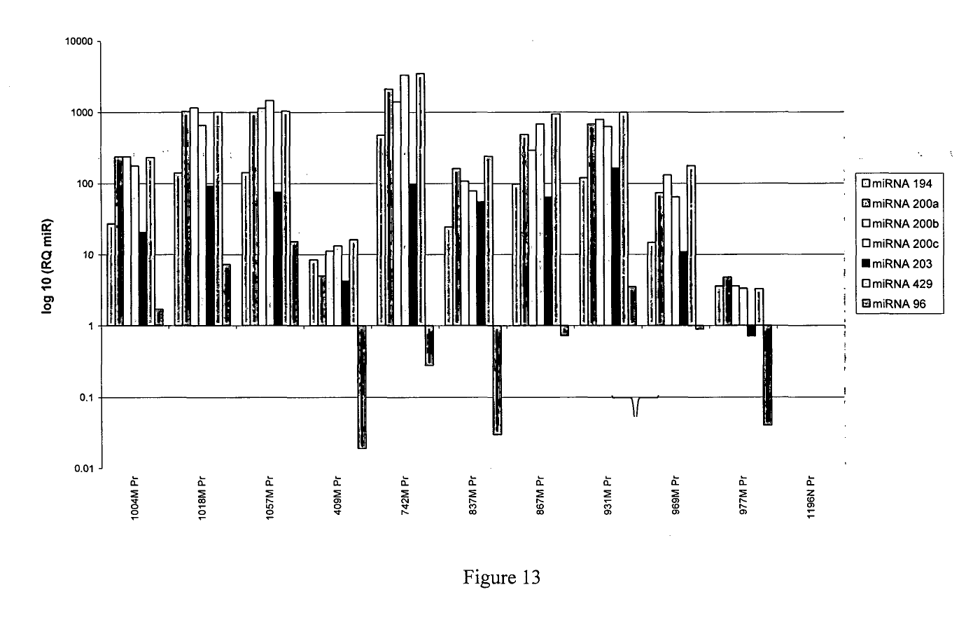

Further, qPCR assays were performed for the 8 microRNA fragments in human tissues obtained from patients operated on for peritoneal metastasis originating from colon cancer. The relative expression of the 8 selected microRNAs was high in all peritoneal metastases compared to normal peritoneum (FIG. 13). The reference calibrator for normal peritoneum control was 1196n Pr (right column).

### Example 9

### Expression of miR-566 in Stool and Colon Tissue

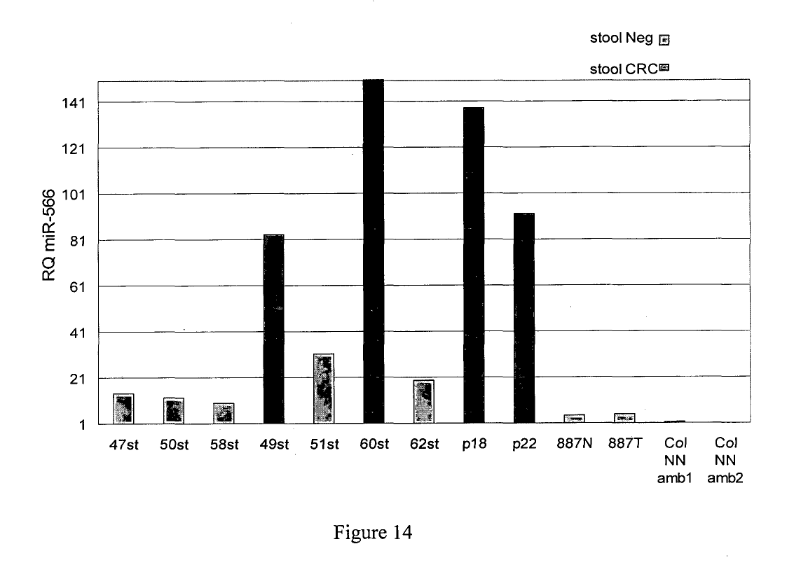

Stool samples were obtained from patients undergoing diagnostic or screening colonoscopy. Stool samples (ST) from patient undergoing colonoscopy with normal findings (dark column) and positive for adenomas (bright column) were compared to stool samples from patients (P) with colon cancer. As seen in FIG. 14, miR566 may be used for diagnosing CRC in stool samples.

### REFERENCES

1. Yong Sun Lee and Anindya Duna, Annu. Rev. Pathol. Mech. Dis. 2009; 4:199-227.
2. J S Ross, A Carlson and Graham Brock, Am J Clin Path 2007: 128; 830-836.
3. Massimo Negrini, Milena S Nicoloso, George A Galin, Current Op. in Cell Biol. 2009: 21; 1-10.
4. Shao-Yao Ying at el., Mol. Biotechnol. 2008: 38; 257-268.
5. S Rossi, C Sevignani, S C Nhadi, L D Siracusa and G A, Mamm Genome 2008: 19(7-8); 526-540.
6. R Garzon, M Fabbri, A Cimmino, G A Galin and C M Croce, Trends in Mol. Med. 2006: 12(12); 580-587.
7. G A Galin and C M Croce, Nat Rev Cancer 2006: 6; 857-866.
8. Jemal A et al. CA Cancer J Clin 2009: 59; 225-249.
9. Li Ma and Robert A. Weinberg, Trends in Genetics 2008: 24(9); 448-456.
10. Carlos E Bosch Roig et al. Clin Transl Oncol 2008: 10; 572-278.
11. T Schepeler et al. Cancer Res 2008: 68(15); 6416-6424.
12. M I Aslam et al., British Jour. of Surgery 2009: 96; 702-710
13. A J Bilchick et., Annals of Surgery 2007: 246; 568-577.
14. A Stojadinovic, A Nissan et., Annals of Surgery 2007; 245(6): 846-857.
15. A Nissan, Journal of Surgical Oncology 2007: 96; 185-187.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for diagnosing metastasis of CRC in a subject, the method comprising determining the expression level of a plurality of miRNAs selected from the group consisting of miR566, miR96, miR183, miR194, miR200a, miR200b, miR200c, miR203 and miR429, or combinations thereof, in a biological sample obtained from the subject, wherein a significant elevation in the expression levels of the plurality of miRNAs in the biological sample compared to control values indicates that said subject is afflicted with metastasis of CRC.

2. The method of claim 1, wherein said plurality of miRNAs consists of miR96, miR183, miR194, miR200a, miR200b, miR200c, miR203 and miR429 or wherein said plurality of miRNAs consists of miR566, miR96, miR183, miR194, miR200a, miR200b, miR200c, miR203 and miR429.

3. (canceled)

4. The method of claim 1 wherein said metastasis is selected from micrometastasis occult metastasis.

5. (canceled)

6. The method of claim 1 for detection of minimal residual CRC.

7. The method of claim 1, wherein said biological sample is selected from the group consisting of tissue, fluid or excretion samples or wherein the biological sample is an excretion sample selected from urine or stool.

8. (canceled)

9. The method of claim 7, wherein the excretion sample is stool or wherein the biological sample is a fluid sample selected from a group consisting of blood, serum, lymph fluid, peritoneal fluid, or lavage of body cavities or organs or wherein the biological sample is a tissue biopsy.

10. (canceled)

11. The method of claim 9, wherein the fluid sample is selected from blood, serum or plasma.

12. (canceled)

13. The method of claim 9, wherein the tissue biopsy is a lymph node biopsy.

14. The method of claim 1, wherein the control values are determined in a sample obtained from said subject, or wherein the control values are selected from the group consisting of values obtained from a healthy subject, a panel of control values from a set of healthy subjects, and a stored set of data corresponding to healthy subjects.

15. The method of claim 14, wherein the sample obtained from said subject is an adjacent normal colonic tissue, the control value correlates to the expression level of the miRNA in the adjacent normal colonic tissue.

16. (canceled)

17. The method of claim 14, wherein said control values correlate to the expression level of the miRNA in a non-cancerous sample selected from the group consisting of normal colonic tissue obtained from a healthy subject, lymph nodes obtained from a healthy subject or peripheral blood lymphocytes of a healthy subject.

18. A method for diagnosing colorectal cancer (CRC) in a subject, the method comprising determining the expression level of a plurality of miRNAs selected from the group consisting of miR566, miR96, miR183, miR194, miR200a, miR200b, miR200c, miR203 and miR429 or combination thereof, in a biological sample obtained from the subject, wherein a significant elevation in the expression levels of the plurality of miRNAs in the biological sample compared to control values indicates that said subject is afflicted with CRC.

19. A method for diagnosing a precancerous lesion in a subject, the method comprising determining the expression level of a plurality of miRNAs selected from the group consisting of miR566, miR96, miR183, miR194, miR200a, miR200b, miR200c, miR203 and miR429 or combination thereof, in a biological sample obtained from the subject, wherein a significant elevation in the expression levels of the plurality of miRNAs in the biological sample compared to control values indicates that said subject is afflicted with a precancerous lesion.

20. The method of claim 19, wherein said precancerous lesion is premalignant colorectal polyps.

21. The method of claim 20, wherein the premalignant colorectal polyps is adenomatous polyp.

22. The method of claim 19, wherein said plurality of miRNAs consists of miR96, miR183, miR194, miR200a, miR200b, miR200c, miR203 and miR429 or wherein said plurality of miRNAs consists of miR566, miR96, miR183, miR194, miR200a, miR200b, miR200c, miR203 and miR429.

23. The method of claim 18, wherein consists of miR96, miR183, miR194, miR200a, miR200b, miR200c, miR203 and miR429, or wherein said plurality of miRNAs consists of miR566, miR96, miR183, miR194, miR200a, miR200b, miR200c, miR203 and miR429.

24. The method of claim 18 for detection of minimal residual CRC.

25. The method of claim 18, wherein the biological sample is a lymph node sample, the method further comprises determining the number of lymph nodes in said subject expressing said plurality of miRNAs, wherein the number of lymph nodes expressing said plurality of miRNAs indicates the stage of a cancer.

26. A method for staging colorectal cancer (CRC) in a subject, the method comprising:
(a) obtaining a plurality of lymph node samples from the subject;
(b) determining the number of lymph nodes having a significant elevation in the a expression level of a plurality of miRNAs compared to control values, wherein the plurality of miRNAs is selected from the group consisting of miR96, miR183, miR194, miR200a, miR200b, miR200c, miR203 and miR429;
wherein the number of lymph nodes having a significant elevation in the expression level of said plurality of miRNAs indicates the stage of CRC.

27. The method of claim 26, wherein said plurality of miRNAs consists of miR96, miR183, miR194, miR200a, miR200b, miR200c, miR203 and miR429.

28. The method of claim 1, wherein determining the expression levels comprises determining the RNA expression levels of said plurality of miRNAs.

29. The method of claim 28, wherein the RNA expression levels are determined by a method selected from nucleic acid hybridization, in situ hybridization (ISH) or nucleic acid amplification, and a combination thereof.

30. The method of claim 29, comprising amplifying RNA extracted from said biological sample.

31. The method of claim 30, wherein amplifying RNA is performed by polymerase chain reaction (PCR).

32. The method of claim 31, wherein the PCR is real-time PCR or wherein said PCR is a quantitative real-time PCR (qRT-PCR).

33. (canceled)

34. (canceled)

35. A kit suitable for use in diagnosing and staging CRC in a subject, comprising means for determining the expression level of a plurality of miRNAs selected from the group consisting of miR566, miR96, miR183, miR194, miR200a, miR200b, miR200c, miR203 and miR429.

36. The kit of claim 35, wherein said plurality of miRNAs consists of miR566, miR96, miR183, miR194, miR200a, miR200b, miR200c, miR203 and miR429 or wherein said plurality of miRNAs consists of miR96, miR183, miR194, miR200a, miR200b, miR200c, miR203 and miR429.

37. (canceled)

38. The kit of claim 35, comprising miRNA hybridization or amplification reagents and at least one probe or amplification primer specific for each member selected from the plurality of miRNAs.


SEQUENCE LISTING (19 sequences)

SEQ ID NO: 1 - DNA, 19 bp, Homo sapiens
gggcgccugugaucccaac

SEQ ID NO: 2 - DNA, 23 bp, Homo sapiens
uuuggcacuagcacauuuuugcu

SEQ ID NO: 3 - DNA, 22 bp, Homo sapiens
uauggcacugguagaauucacu

SEQ ID NO: 4 - DNA, 22 bp, Homo sapiens
uguaacagcaacuccaugugga

SEQ ID NO: 5 - DNA, 22 bp, Homo sapiens
uaacacugucugguaacgaugu

SEQ ID NO: 6 - DNA, 22 bp, Homo sapiens
uaauacugccugguaaugauga

SEQ ID NO: 7 - DNA, 23 bp, Homo sapiens
uaauacugccggguaaugaugga

SEQ ID NO: 8 - DNA, 22 bp, Homo sapiens
gugaaauguuuaggaccacuag

SEQ ID NO: 9 - DNA, 22 bp, Homo sapiens
uaauacugucugguaaaaccgu

SEQ ID NO: 10 - DNA, 94 bp, Homo sapiens
gcuaggcgugguggcgggcgccugugaucccaacuacucaggaggcuggggcagcagaaucgcuugaacccgggaggcgaagguugcagugagc

SEQ ID NO: 11 - DNA, 78 bp, Homo sapiens
uggccgauuuuggcacuagcacauuuuugcuugugucucuccgcucugagcaaucaugugcagugccaauaugggaaa

SEQ ID NO: 12 - DNA, 110 bp, Homo sapiens
ccgcagagugugacuccuguucuguguauggcacugguagaauucacugugaacagucucagucagugaauuaccgaagggccauaaacagagcagagacagauccacga

SEQ ID NO: 13 - DNA, 85 bp, Homo sapiens
augguguuaucaaguguaacagcaacuccauguggacuguguaccaauuuccaguggagaugcuguuacuuuugaugguuaccaa

SEQ ID NO: 14 - DNA, 90 bp, Homo sapiens
ccgggccccugugagcaucuuaccggacagugcuggauuucccagcuugacucuaacacugucugguaacgauguucaaaggugacccgc

SEQ ID NO: 15 - DNA, 95 bp, Homo sapiens
ccagcucgggcagccguggccaucuuacugggcagcauuggauggagucaggucucuaauacugccugguaaugaugacggcggagcccugcacg

SEQ ID NO: 16 - DNA, 68 bp, Homo sapiens
cccucgucuuacccagcaguguuugggugcgguugggagucucuaauacugccggguaaugauggagg

SEQ ID NO: 17 - DNA, 110 bp, Homo sapiens
guguuggggacucgcgcgcuggguccagugguucuuaacaguucaacaguucuguagcgcaauugugaaauguuuaggaccacuagacccggcgggcgcggcgacagcga

SEQ ID NO: 18 - DNA, 83 bp, Homo sapiens
cgccggccgaugggcgucuuaccagacaugguuagaccuggcccucugucuaauacugucugguaaaaccguccauccgcugc

SEQ ID NO: 19 - DNA, 2544 bp, Homo sapiens
gccttaatagctagctggatgaatgtttaacttctaggccaggcactactctgtcccaacaataagccctgtacattgggaaaggtgccgagacatgaactttggtcttctctgcaatccatctggagcattcactgacaacatcgactttgaagttgcactgacctggccagccctgccacttaccaggttggctctgtatggctaagcgttttctcctaaaatcccttgaaaactgtgagaagaccataagaagatcatatctttaattctatttcacaagtcacacaatattccaatcaaatacagatggttgagaaaagtcatccatcttccctccccaccctcccacagcccctcaaccactgccctgaaacttatatgctgttatccgcagctccatctggagcatcacagctactgtcaaccctgacgctctttctgaaaaaacaccggatggacatcagaactatttctttaaggatgttactgagccacacaggaaaacttgccttatgattttgaatgcacggatctgatttgactaaacatgataactagagaatcacccaatctactcccattttcaactctaaatcatcagagtgtctcaaatccaaagcacacacagaccagcctggccaacacggtgaaactccacccctactaaaagtataaaaattatccaggtgtggtggcgggcgcctgtaatccaagctacttgggagtctggaggcaggagaatcccttgaacctgggagatggaggttgcagtgagcagagatcacaccaccgcactctagcctgggccacaaatcaacaacaacaacaacaacaaaaaacaaagcgcacacagagactgaggtcctctttggcattgagaagatggctatgcaagtcccaactagcaagtgcaaacttcccagcttcacttctgccagtgtcccttcaccccttctcaaccccactgggaggcaggagggtgcttgacaataacagccttggcatcactctgccagggtgtaataggaactgttacaattctgagattctgtgtaagcactggcctttctgcctagaatgccttctcctctcttttttaactgcatgctcctatttatctttcaaagcccggaaaaaataacactgcacacgggaaatgctcccttcctactgcagtcatttagatgactctatgccattccattcatttctctttcctaccacagaagtgctttgagattttggagtcagactgcttgaacttgaatcctggccctctcatcagagacttgacttattttaggcaagttatataaccaattttacctcagttccttacccataaaatgggtctaatgagagtacctaccacacagaattttgatgaaaactgaatgagatgaaggcctttaaggcagtggtccccaaccctggggacacagacaggtaccattttgtggcctgttaggaactgggccacacagcaggaggtgagcagtgggtgagtgagatcagcgttatttacagctgctccccattgctcaccttactgcctgagctccacctcctgtcagatcagcagtggcattaaattctcatagcagcacaaaccctgtcatgaactgcacatgcgagggatctaggttgtgcgctccttatgagaatctaatgcctaatgacctgtcaccgtctcccatcacccctagatgggagtgtctagttgcaggaaacaagctcagggcttccactgattctacattatggtgagttgtataattatttcattatataatacaatgtaataataatagaaacacagtgcacaacaaatgtaatgtgcttgaatcatccccaaaccatcccagtccacggtcttccacattttgtcttttcacaaaattgtcttccacaaaactggtccctggtgccaaaaaggcttgggaccactgctttaaagcctttgcatagtgcttagaattgagggggaaaaaaaaaacaaaaacaatgtagctagttgctacaatcactatattggtgagtttcaaaaggaaaagaattctgtcccatttatgcttgagccttgagttgctaaccaagcctgacacaaaattactgttgaagggatgtgtgagtcctaattgaaatgaggcctcttaagggaattgtggaccaaaccccaagcaggcagaaagccgtatcttaattattgcaagtatttcaggcaaggtgtggatggccatttgaattcaagcagactaggacctgggatgagaaagaaggtgtgtacgtgacttgatctttgaactttagctcaccatctggaagaaggctgagtattctctgcactcacatagtagctaatgcctactccccagccacccacaattctttctgtaggaaggctcgctagaatactttgtgatattggatattagttccatattctactgtgtatcttagttcaaccaaattgtaatcatctgatatttatttcttttaatataaatataagtatattaagtcttaaaaaaaaaaaaaaaa